(19) United States
(12) Patent Application Publication
(10) Pub. No.: US 2005/0143384 A1
(43) Pub. Date: Jun. 30, 2005

(54) Amide thiadiazole inhibitors of plasminogen activator inhibitor-1

(76) Inventors: Eric Sartori, Paris (FR); Magali Maillet, Suresne (FR); Marie France Paugam, Fontenay le Fleury (FR); Eric Nicolai, Rueil-Malmaison (FR); Michael Lawrence, Yardley, PA (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/969,692

(22) Filed: Oct. 20, 2004

Related U.S. Application Data
(60) Provisional application No. 60/515,898, filed on Oct. 30, 2003.

(51) Int. Cl. C07D 417/02; C07D 285/14; A61K 31/501; A61K 31/4439; A61K 31/433

(52) U.S. Cl. 514/252.05; 514/341; 514/362; 544/238; 546/268.7; 548/138

(57) ABSTRACT

Methods of treating disorders associated with elevated levels of PAI-1 are disclosed comprising administering to a patient in need thereof a therapeutically effective amount of at least one compound of formula at least one compound of formula (I), 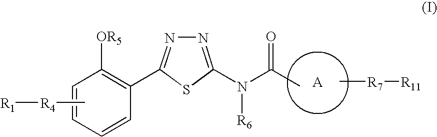
or a pharmaceutically-acceptable salt, prodrug, stereoisomer or solvate thereof, wherein: A is aryl or heteroaryl and R₁—R₁₁, are defined herein. The invention also pertains to pharmaceutical compositions and compounds within the scope of formula (I) as well as medicaments and articles of manufacture comprising compounds of formula (I).

This application claims the benefit of priority from U.S. Provisional Application Ser. No. 60/515,898, filed Oct. 30, 2003.

### FIELD OF THE INVENTION

This invention relates to methods of using amide thiadiazole compounds to inhibit Plasminogen Activator Inhibitor-1 (“PAI-1”) to treat disorders associated with elevated levels of PAI-1, as well as pharmaceutical compositions and articles of manufacture comprising the amide thiadiazole compounds.

### BACKGROUND OF THE INVENTION

Plasminogen activator inhibitor-1 (“PAI-1”) is a member of the serine protease inhibitor (SERPIN) superfamily of proteins and plays a major role in the regulation of the plasminogen-plasmin system. PAI-1 is known to be the principal inhibitor of the serine proteases tissue-type plasminogen activator (t-PA) and urokinase-type plasminogen activator (u-PA). In the plasma, t-PA cleaves the zymogen plasminogen to the active enzyme plasmin, which can degrade fibrin clots (fibrinolysis or thrombolysis) thereby exerting an antithrombotic effect. Therefore, through the regulation of t-PA, PAI-1 plays an important role in hemostasis. An increased level of PAI-1 is believed to be a risk factor in thrombotic conditions such as venous thrombosis, atherosclerosis, and arterial thrombosis, which can result in deep vein thrombosis, pulmonary embolism, myocardial infarction, stroke, etc. See e.g., Wu, Current Drug Targets, 2, 27, (2002); Dawson, et al., Atherosclerosis, 95, 105 (1992); Wiman, et al., Thrombosis and Haemostasis, 74, 71, (1995); V. Salomaa, et al., Circulation, 91, 284 (1995); and Eitzman, Blood, 96, 4212 (2000). In animal experiments, inhibitors of PAI-1 activity have been shown to be effective at treating thrombotic conditions. See e.g., Berry, et al, British Journal of Pharmacology, 125, 29 (1998) and Friedrich, et al., Circulation, 96, 916, (1997).

In tissue matrices, u-PA converts plasminogen to plasmin which activates matrix metalloproteases (MMPs) that degrade extracellular matrix (ECM). Through this regulation of u-PA, PAI-1 therefore plays an important role in cellular migration and tissue remodelling processes. PAI-1 is thus believed to modulate diseases and conditions such as wound healing, angiogenesis, cancer invasion, and metastasis. Increased levels of PAI-1 have been associated with poor prognosis in cancer patients (T. L. Frandesen, Drugs Future, 873, 1998; Pappot, et al, Biol. Chem. Hoppe-Seyler, 376, 259, 1995). PAI-1 has additionally been associated with other conditions and diseases such as obesity and insulin resistance (Juhan-Vague, et al, Journal of Thrombosis and Haemostasis, 1, 1575, 2003), inflammatory diseases, such as asthma (Cho, et al. Journal of Allergy and Clinical Immunology, 108, 212, 2001), and renal disease (Brown, et al, Journal of Nephrology, 15, 230, 2002). See also Tsikouris et al., J. Clin. Pharmacol., 42:1187,2002 and Binder et al., News Physiol, Sci. 17,56, 2002.

Accordingly, compounds that inhibit PAI-1 would be useful in the treatment of several disease states and disorders, especially thromboembolic disorders.

### SUMMARY OF THE INVENTION

The instant invention pertains to methods of inhibiting PAI-1 inhibitors comprising administering to a patient in need thereof a therapeutically effective amount of at least one compound of formula (I), at least one compound of formula (I), 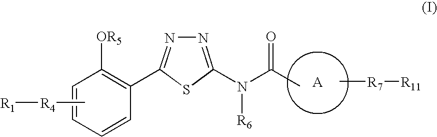
or a pharmaceutically-acceptable salt, prodrug, stereoisomer or solvate thereof, wherein:
A is aryl or heteroaryl;
R₁-R₄ and R₇-R₁₁ are independently selected from hydrogen, halogen, nitro, cyano, alkyl, substituted alkyl, aryl, heteroaryl, cycloalkyl, heterocyclo, —OR₁₂, —SR₁₂, —OC(═O)R₁₂, —C(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₃C(═O)R₁₂, —NR₁₅CO₂R₁₂, —NR₁₅C(═O)NR₁₃R₁₄;
or any two of R₁-R₄ and R₇-R₁₁ located on neighboring atoms of the ring to which they are attached may be taken together to form a fused ring system in combination with the ring, wherein the fused ring system may be optionally further substituted;
R₅ is hydrogen, C₁₋₆alkyl or substituted C₁₋₆alkyl;
R₆is hydrogen or C₁₋₆alkyl; and
R₁₂, R₁₃, R₁₄ and R₁₅ are independently selected from hydrogen, alkyl, subtituted alkyl, aryl, heteroaryl, cycloalkyl and heterocyclo, wherein each instance of R₁₂, R₁₃, R₁₄ and/or R₁₅ is selected independently.

The invention further relates to compounds having formula (Ia), 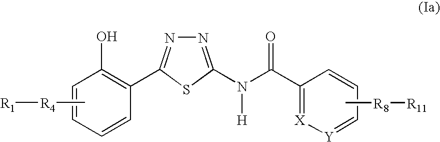
or a pharmaceutically-acceptable salt, prodrug, stereoisomer or solvate thereof, wherein:
X and Y are independently carbon or nitrogen;
R₁ to R₄ are independently selected from hydrogen, halogen, nitro, cyano, alkyl, substituted alkyl, —OR₁₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₃C(═O)R₁₂, —NR₁₅CO₂R₁₂, —NR₁₅C(═O)NR₁₃R₁₄;
R₈-R₁₁ are independently selected from hydrogen, halogen, nitro, cyano, alkyl, substituted alkyl, aryl, heteroaryl, cycloalkyl, heterocyclo, —OR₁₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₅C(═O)R₁₂, —NR₁₅CO₂R₁₂, —NR₁₅C(═O)NR₁₃R₁₄; and
or any two of R₈-R₁₁ located on neighboring atoms of the ring may be taken together to form a fused ring system in combination with the ring, said fused ring system being optionally substituted; and
R₁₂, R₁₃, R₁₄ and R₁₅ are independently selected from hydrogen, alkyl, substituted alkyl, aryl, heteroaryl, cycloalkyl and heterocyclo, wherein each instance of R₁₂, R₁₃, R₁₄ and/or R₁₅ is selected independently.

The invention also pertains to pharmaceutical compositions within the scope of formula (I) as well as medicaments and articles of manufacture comprising compounds of formula (I).

### DESCRIPTION OF THE INVENTION

Listed below are definitions of various terms used to describe this invention. These definitions apply to the terms as they are used throughout this specification, unless otherwise limited in specific instances, either individually or as part of a larger group.

The term “alkyl” refers to straight or branched chain unsubstituted hydrocarbon groups of 1 to 20 carbon atoms, preferably 1 to 7 carbon atoms. The expression “lower alkyl” refers to unsubstituted alkyl groups of 1 to 4 carbon atoms. When a subscript is used with reference to an alkyl or other group, the subscript refers to the number of carbon atoms that the group may contain. For example, the term “C₀₋₄alkyl” includes a bond and alkyl groups of 1 to 4 carbon atoms.

The term “substituted alkyl” refers to an alkyl group substituted by one to four substituents selected from halogen, hydroxy, alkoxy, keto (═O), alkanoyl, aryloxy, alkanoyloxy, NRₐR_{b}, alkanoylamino, aroylamino, aralkanoylamino, substituted alkanoylamino, substituted arylamino, substituted aralkanoylamino, thiol, alkylthio, arylthio, aralkylthio, alkylthiono, arylthiono, aralkylthiono, alkylsulfonyl, arylsulfonyl, aralkylsulfonyl, —SO₂NRₐR_{b}, nitro, cyano, —CO₂H, —CONRₐR_{b}, alkoxycarbonyl, aryl, guanidino and heteroaryls or heterocyclos (such as indolyl, imidazolyl, furyl, thienyl, thiazolyl, pyrrolidyl, pyridyl, pyrimidyl and the like), wherein Rₐ and R_{b} are selected from hydrogen, alkyl, aryl, aralkyl, cycloalkyl, cycloalkylalkyl, heteroaryl, heteroarylalkyl, heterocycle, and heterocyclealkyl. The substituent on the alkyl optionally in turn may be further substituted, in which case it will be with substituted one or more of C₁₋₄alkyl, C₂₋₄alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenyloxy, and/or benzyloxy.

The term “alkenyl” refers to straight or branched chain hydrocarbon groups of 2 to 20 carbon atoms, preferably 2 to 15 carbon atoms, and most preferably 2 to 8 carbon atoms, having at least one double bond, and depending on the number of carbon atoms, up to four double bonds.

The term “substituted alkenyl” refers to an alkenyl group substituted by one to two substituents selected from those recited above for substituted alkyl groups.

The term “alkynyl” refers to straight or branched chain hydrocarbon groups of 2 to 20 carbon atoms, preferably 2 to 15 carbon atoms, and most preferably 2 to 8 carbon atoms, having at least one triple bond, and depending on the number of carbon atoms, up to four triple bonds.

The term “substituted alkynyl” refers to an alkynyl group substituted by one to two substituents selected from those recited above for alkyl groups.

When the term alkyl is used in connection with another group, as in heterocycloalkyl or cycloalkylalkyl, this means the identified (first named) group is bonded directly through an alkyl group which may be branched or straight chain (e.g., cyclopropylC₁₋₄alkyl means a cyclopropyl group bonded through a straight or branched chain alkyl group having one to four carbon atoms.). In the case of substituents, as in “substituted cycloalkylalkyl,” the alkyl portion of the group, besides being branched or straight chain, may be substituted as recited above for substituted alkyl groups and/or the first named group (e.g., cycloalkyl) may be substituted as recited herein for that group.

The term “halogen” or “halo” refers to fluorine, chlorine, bromine and iodine.

The term “aryl” refers to monocyclic or bicyclic aromatic substituted or unsubstituted hydrocarbon groups having 6 to 12 carbon atoms in the ring portion, such as phenyl, naphthyl, and biphenyl groups. Each ring of the aryl may be optionally substituted with one to three R_{c} groups, wherein R_{c} at each occurrence is selected from alkyl, substituted alkyl, halogen, trifluoromethoxy, trifluoromethyl, —SR, —OR, —NRR′, —NRSO₂R′, —SO₂R, —SO₂NRR′, —CO₂R′, —C(═O)R′, —C(═O)NRR′, —OC(═O)R′, —OC(═O)NRR′, —NRC(═O)R′, —NRCO₂R′, phenyl, C₃₋₇ cycloalkyl, and five-to-six membered heterocyclo or heteroaryl, wherein each R and R′ is selected from hydrogen, alkyl, substituted alkyl, alkenyl, substituted alkenyl, phenyl, C₃₋₇cycloalkyl, and five-to-six membered heterocyclo or heteroaryl, except in the case of a sulfonyl group, then R is not going to be hydrogen. Each substituent R_{c} optionally in turn may be further substituted by one or more (preferably 0 to 2) R_{d} groups, wherein R_{d} is selected from C₁₋₆alkyl, C₂₋₆alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenylethyl, phenyloxy, and benzyloxy.

The term “aralkyl” refers to an aryl group bonded directly through an alkyl group, such as benzyl, wherein the alkyl group may be branched or straight chain. In the case of a “substituted aralkyl,” the alkyl portion of the group besides being branched or straight chain, may be substituted as recited above for substituted alkyl groups and/or the aryl portion may be substituted as recited herein for aryl. Thus, the term “optionally substituted benzyl” refers to the group 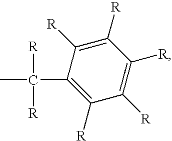
wherein each R group may be hydrogen or may also be selected from R_{c} as defined above, in turn optionally substituted with one or more R_{d}. At least two of these “R” groups should be hydrogen and preferably at least five of the “R” groups is hydrogen. A preferred benzyl group involves the alkyl-portion being branched to define

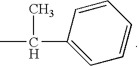

The term “heteroaryl” refers to a substituted or unsubstituted aromatic group for example, which is a 4 to 7 membered monocyclic, 7 to 11 membered bicyclic, or 10 to 15 membered tricyclic ring system, which has at least one heteroatom and at least one carbon atom-containing ring. Each ring of the heteroaryl group containing a heteroatom can contain one or two oxygen or sulfur atoms and/or from one to four nitrogen atoms, provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. It may optionally be substituted with one to three (preferably 0 to 2) R_{c} groups, as defined above for aryl, which in turn may be substituted with one or more (preferably o to 2) R_{d} groups, also as recited above.

Exemplary monocyclic heteroaryl groups include pyrrolyl, pyrazolyl, pyrazolinyl, imidazolyl, oxazolyl, isoxazolyl, thiazolyl (i.e., 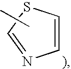
thiadiazolyl, isothiazolyl, furanyl, thienyl, oxadiazolyl, pyridyl, pyrazinyl, pyrimidinyl, pyridazinyl, triazinyl, tetrazinyl, and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzirnidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

The term “cycloalkyl” refers to a saturated or partially unsaturated non-aromatic cyclic hydrocarbon ring system, preferably containing 1 to 3 rings and 3 to 7 carbon atoms per ring, which may be substituted or unsubstituted and/or which may be fused with a C₃-C₇ carbocylic ring, a heterocyclic ring, or which may have a bridge of 3 to 4 carbon atoms. The cycloalkyl groups including any available carbon or nitrogen atoms on any fused or bridged rings optionally may have 0 to 3 (preferably 0-2) substituents selected from R_{c} groups, as recited above, and/or from keto (where appropriate) which in turn may be substituted with one to three R_{d} groups, also as recited above. Thus, when it is stated that a carbon-carbon bridge may be optionally substituted, it is meant that the carbon atoms in the bridged ring optionally may be substituted with an R_{c} group, which preferably is seleted from C₁₋₄alkyl, C₂₋₄alkenyl, halogen, haloalkyl, haloalkoxy, cyano, amino, C₁₋₄alkylamino, aminoC₁₋₄alkyl, hydroxy, hydroxyC₁₋₄alkyl, and C₁₋₄alkoxy. Exemplary groups include cyclopropyl, cyclobutyl, cyclopentyl, cyclohexyl, cycloheptyl, bicycloheptane, cycloctyl, cyclodecyl, cyclododecyl, and adamantyl.

The terms “heterocycle”, “heterocyclic” and “heterocyclo” each refer to a fully saturated or partially unsaturated nonaromatic cyclic group, which may be substituted or unsubstituted, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen, oxygen, and sulfur atoms, where the nitrogen and sulfur heteroatoms also optionally may be oxidized and the nitrogen heteroatoms also optionally may be quaternized. Preferably two adjacent heteroatoms are not simultaneously selected from oxygen and nitrogen. The heterocyclic group may be attached at any nitrogen or carbon atom. The heterocyclo groups optionally may have 0 to 3 (preferably 0-2) substituents selected from keto (═O), and/or one or more R_{c} groups, as recited above, which in turn may be substituted with one to three R_{d} groups, also as recited above.

Exemplary monocyclic heterocyclic groups include pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, dioxanyl, isothiazolidinyl, thietanyl, thiiranyl, triazinyl, and triazolyl, and the like.

Exemplary bicyclic hetrocyclic groups include 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl] or furo[2,3-b]pyridinyl), dihydroisoindolyl, dihydroquinazolinyl (such as 3,4-dihydro-4-oxo-quinazolinyl), benzisothiazolyl, benzisoxazolyl; benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like.

Also included are smaller heterocyclos, such as epoxides and aziridines.

Unless otherwise indicated, when reference is made to a specifically-named aryl (e.g., phenyl), cycloalkyl (e.g., cyclohexyl), heterocyclo (e.g., pyrrolidinyl) or heteroaryl (e.g., indolyl), the reference is intended to include rings having 0 to 3, preferably 0-2, substituents selected from those recited above for the the aryl, cycloalkyl, heterocyclo and/or heteroaryl groups, as appropriate. Additionally, when reference is made to a specific heteroaryl or heterocyclo group, the reference is intended to include those systems having the maximum number of non-cumulative double bonds or less than the maximum number of double bonds. Thus, for example, the term “isoquinoline” refers to isoquinoline and tetrahydroisoquinoline.

Additionally, it should be understood that one skilled in the field may make appropriate selections for the substituents for the aryl, cycloalkyl, heterocyclo, and heteroaryl groups to provide stable compounds and compounds useful as pharmaceutically-acceptable compounds and/or intermediate compounds useful in making pharmaceutically-acceptable compounds. Thus, for example, in compounds of formula (I), when a substituent is a cyclopropyl ring, preferably the ring has no more than two substituents, and preferably said substituents do not comprise nitro (NO₂), more than one cyano group, or three halogen groups.

The term “heteroatoms” shall include oxygen, sulfur and nitrogen.

The term “haloalkyl” means an alkyl having one or more halo substituents.

The term “perfluoromethyl” means a methyl group substituted by one, two, or three fluoro atoms, i.e., CH₂F, CHF₂ and CF₃. The term “perfluoroalkyl” means an alkyl group having from one to five fluoro atoms, such as pentafluoroethyl.

The term “haloalkoxy” means an alkoxy group having one or more halo substituents. For example, “haloalkoxy” includes —OCF₃.

The term “carbocyclic” means a saturated or unsaturated monocyclic or bicyclic ring in which all atoms of all rings are carbon. Thus, the term includes cycloalkyl and aryl rings. The carbocyclic ring may be substituted in which case the substituents are selected from those recited above for cycloalkyl and aryl groups.

When the term “unsaturated” is used herein to refer to a ring or group, the ring or group may be fully unsaturated or partially unsaturated.

Definitions for the various other groups that are recited above in connection with substituted alkyl, substituted alkenyl, aryl, cycloalkyl, and so forth, are as follows: alkoxy is —OR^{e}, alkanoyl is —C(═O)R^{e}, aryloxy is —OAr, alkanoyloxy is —OC(═O)R^{e}, amino is —NH₂, alkylamino is —NHR^{e} or —N(R^{e})₂, arylamino is —NHAr or —NR^{e}Ar, aralkylamino is —NH—R^{f}—Ar, alkanoylamino is —NH—C(═O)R^{e}, aroylamino is —NH—C(═O)Ar, aralkanoylamino is —NH—C(═O)R^{f}—Ar, thiol is —SH, alkylthio is —SR^{e}, arylthio is —SAr, aralkylthio is —S—R^{f}—Ar, alkylthiono is —S(═O)R^{e}, arylthiono is —S(═O)Ar, aralkylthiono is —S(═O)R^{f}—Ar, alkylsulfonyl is —SO_{(q)}R^{e}, arylsulfonyl is —SO_{(q)}Ar, arylsulfonylamine is —NHSO_{(q)}Ar, alkylsulfonylamine is —NHSO₂R^{e}, aralkylsulfonyl is —SO_{(q)}R^{f}Ar, sulfonamido is —SO₂NH₂, substituted sulfonamide is —SO₂NHR^{e} or —SO₂N(R^{e})₂, nitro is —NO₂, carboxy is —CO₂H, carbamyl is —CONH₂, substituted carbamyl is —C(═O)NHR^{g} or —C(═O)NR^{g}R^{h}, alkoxycarbonyl is —C(═O)OR^{e}, carboxyalkyl is —R^{f}—CO₂H, sulfonic acid is —SO₃H, arylsulfonylamine is —NHSO_{(q)}Ar, guanidino is 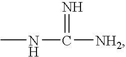
and ureido is 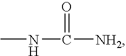
wherein R^{e} is alkyl or substituted alkyl as defined above, R^{f} is alkylene or substituted alkylene as defined above, R^{g} and R^{h} are selected from alkyl, substituted alkyl, aryl, aralkyl, cycloalkyl, heterocyclo, and heteraryl; Ar is an aryl as defined above, and q is 2 or 3.

Throughout the specification, groups and substituents thereof may be chosen by one skilled in the field to provide stable moieties and compounds.

The compounds of Formula (I) may form salts which are also within the scope of this invention. Pharmaceutically acceptable (i.e. non-toxic, physiologically acceptable) salts are preferred, although other salts are also useful, e.g., in isolating or purifying the compounds of this invention.

The compounds of Formula (I) may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for Formula (I) may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art. Salt forms of the compounds may be advantageous for improving the compound dissolution rate and oral bioavailability.

In addition, zwitterions (“inner salts”) may be formed.

All stereoisomers of the compounds of the instant invention are contemplated, either in admixture or in pure or substantially pure form. The definition of compounds according to the invention embraces all the possible stereoisomers and their mixtures. It embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

Compounds of the Formula (I) may also have prodrug forms. Any compound that will be converted in vivo to provide the bioactive agent (i.e., the compound for formula I) is a prodrug within the scope and spirit of the invention.

Various forms of prodrugs are well known in the art. For examples of such prodrug derivatives, see:
a) Design of Prodrugs, edited by H. Bundgaard, (Elsevier, 1985) and Methods in Enzymology, Vol.42, p. 309-396, edited by K. Widder, et al. (Acamedic Press, 1985);
b) A Textbook of Drug Design and Development, edited by Krosgaard-Larsen and H. Bundgaard, Chapter 5, “Design and Application of Prodrugs,” by H. Bundgaard, p. 113-191 (1991); and
c) H. Bundgaard, Advanced Drug Delivery Reviews, 8, 1-38 (1992), each of which is incorporated herein by reference.

It should further be understood that solvates (e.g., hydrates) of the compounds of Formula (I) are also with the scope of the present invention. Methods of solvation are generally known in the art.

### Preferred Methods and Compounds

Preferred methods of treating a disorder associated with high levels of PAI-1 are those comprising administering an effective amount of a patient in need of such treatment at least one compound within the scope of formula (I), depicted above, having the formula (Ia), 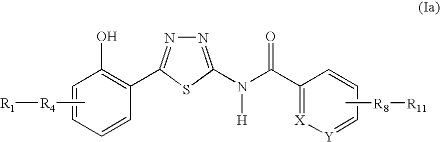
or a pharmaceutically-acceptable salt, prodrug, stereoisomer or solvate thereof, wherein:
X and Y are independently carbon or nitrogen; (preferably X and Y are not both N);
R₁ to R₄ are independently selected from hydrogen, halogen, nitro, cyano, alkyl, substituted alkyl, —OR₁₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₃C(═O)R₁₂, —NR₁₅CO₂R₁₂, —NR₁₅C(═O)NR₁₃R₁₄;
R₈-R₁₁ are independently selected from hydrogen, halogen, nitro, cyano, alkyl, substituted alkyl, aryl, heteroaryl, cycloalkyl, heterocyclo, —OR₁₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₅C(═O)R₁₂, —NR₁₅CO₂R₁₂, —NR₁₅C(═O)NR₁₃R₁₄; and
or any two of R₈-R₁₁ located on neighboring atoms of the ring may be taken together to form a fused ring system in combination with the ring, said fused ring system being optionally substituted.

More preferred methods of treating a disorder associated with high levels of PAI-1 are those comprising administering an effective amount of a patient in need of such treatment at least one compound within the scope of formula (Ia) having the formula (Ib) wherein at least one and preferably all of the variables are chosen from the list below:

Preferred compounds of the present invention correspond to compounds within the scope of formula (Ia), depicted above, having formula (Ib), depicted below, wherein at least one and preferably all of the variables are chosen from the list below: 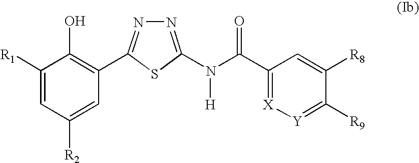
or a pharmaceutically-acceptable salt, prodrug, stereoisomer or solvate thereof, wherein:
R₁ and R₂ are independently selected from halogen, nitro, cyano, C₁₋₆alkyl and C₁₋₆substituted alkyl (preferably R₁ and R₂ are independently halogen); and
R₈ and R₉ are independently selected from hydrogen, halogen, nitro, cyano, alkyl, substituted alkyl, aryl, heteroaryl, heterocyclo, —OR₁₂, —SR₁₂, —C(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅CO₂R₁₂, and —NR₁₅C(═O)R₁₂ (preferably bromo, chloro, iodo, nitro, C₁₋₆alkyl, —OH, heteroaryl (especially pyrrolyl), —O(C₁₋₆alkyl), —CO₂(hydrogen, C₁₋₆alkyl or aryl), —C(═O)NR₁₃R₁₄, —NHCO₂(C₁₋₆alkyl), and —NHC(═O)(aryl or C₁₋₆alkyl, wherein R₁₃ and R₁₄ are preferably hydrogen and aryl),
or R₈ and R₉ may be taken together to form a fused ring system in combination with the ring, said fused ring system being optionally substituted (preferred fused ring systems are dioxo heterocyclo groups);
R₁₂ and R₁₃ are independently selected from hydrogen, C₁₋₆alkyl, substituted C₁₋₆alkyl, aryl and heteroaryl; and
R₁₄ and R₁₅ are independently selected from hydrogen, C₁₋₆alkyl and substituted C₁₋₆alkyl.

Even more preferred methods of treating a disorder associated with high levels of PAI-1 are those comprising administering an effective amount of a patient in need of such treatment at least one compound within the scope of formula (Ib) having the formula (Ic) wherein at least one and preferably all of the variables are chosen from the list below:

Preferred compounds of the present invention correspond to compounds within the scope of formula (Ib), depicted above, having formula (Ic), depicted below, wherein at least one and preferably all of the variables are chosen from the list below: 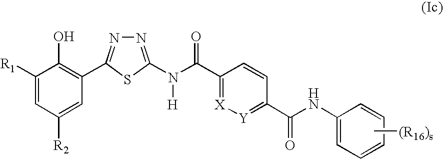
or a pharmaceutically-acceptable salt, prodrug, stereoisomer or solvate thereof, wherein:
R₁₆ is selected from hydrogen, halogen, nitro, cyano, C₁₋₆alkyl, substituted C₁₋₆alkyl, —OR₁₇, —SR₁₇, C(O)R₁₇, —S(═O)R₁₇, —CO₂R₁₇, —C(═O)NR₁₈R₁₉, —NR₁₈R₁₉, —S(═O)R₁₇, —SO₂R₁₇, —SO₂NR₁₈R₁₉ and —NR₂₀C(═O)R₁₇, aryl and heteroaryl (especially preferably halogen, nitro, cyano, OH, C₁₋₆alkyl, —O(C₁₋₆alkyl) substituted by hydrogen or phenyl, —S(C₁₋₆alkyl), —C(O)(C₁₋₆alkyl or phenyl) —CO₂(hydrogen or C₁₋₆alkyl), aryl (including phenyl substituted with hydrogen or halogen), —Ophenyl (especially substituted with hydrogen, halogen, C₁₋₆alkyl, —CO₂(hydrogen or C₁₋₆alkyl), —C(═O)NH₂, and heteroaryl (including five membered rings containing at least one heteroatom selected from N or O) substituted by hydrogen, halogen, C₁₋₆alkyl, —CO₂(hydrogen or C₁₋₆alkyl))
R₁₇ is hydrogen halogen, C₁₋₆alkyl, aryl or heteroaryl;
R₁₈, and R₁₉ are independently selected from hydrogen, C₁₋₆alkyl, aryl or heteroaryl;
   wherein each instance of R₁₆-R₁₉ is selected independently; and s is an integer selected from 1-3.

More preferred compounds of the present invention are selected from the following:
(i) N-[5-(3,5-Dichloro-2-hydroxy-phenyl)-[1,3,4]thiadiazol-2-yl]-terephthalamic acid methyl ester;
N-[5-(3,5-Dichloro-2-hydroxy-phenyl)-[1,3,4]thiadiazol-2-yl]-terephthalamic acid;
N-[5-(3,5-Dichloro-2-hydroxy-phenyl)-[1,3,4]thiadiazol-2-yl]-N′-phenyl-terephthalamide;
N-[5-(3,5-Dichloro-2-hydroxy-phenyl)-[1,3,4]thiadiazol-2-yl]-N′-(4-phenoxy-phenyl)-terephthalamide;
4-(4-{4-[5-(3,5-Dichloro-2-hydroxy-phenyl)-[1,3,4]thiadiazol-2-ylcarbamoyl]-benzoylamino}-phenoxy)-benzoic acid methyl ester;
4-(4-{4-[5-(3,5-Dichloro-2-hydroxy-phenyl)-[1,3,4]thiadiazol-2-ylcarbamoyl]-benzoylamino}-phenoxy)-benzoic acid;
3-{4-[5-(3,5-Dichloro-2-hydroxy-phenyl)-[1,3,4]thiadiazol-2-ylcarbamoyl]-benzoylamino}-benzoic acid ethyl ester;
N-(4-Chloro-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-fluoro-phenyl)-terephthalamide;
N-[5-(3,5-Dichloro-2-hydroxy-phenyl)-[1,3,4]thiadiazol-2-yl]-N′-(4-fluoro-phenyl)-terephthalamide;
N-[5-(3,5-Dichloro-2-hydroxy-phenyl)-[1,3,4]thiadiazol-2-yl]-N′-(9-ethyl-9H-carbazol-3-yl)-terephthalamide;
N-[5-(3,5-Dichloro-2-hydroxy-phenyl)-[1,3,4]thiadiazol-2-yl]-N′-(4-pentyloxy-phenyl)-terephthalamide;
N-[5-(3,5-Dichloro-2-hydroxy-phenyl)-[1,3,4]thiadiazol-2-yl]-N′-(4-phenylamino-phenyl)-terephthalamide;
2-{4-[5-(3,5-Dichloro-2-hydroxy-phenyl)-[1,3,4]thiadiazol-2-ylcarbamoyl]-benzoylamino}-benzoic acid methyl ester;
N-[5-(3,5-Dichloro-2-hydroxy-phenyl)-[1,3,4]thiadiazol-2-yl]-N′-p-tolyl-terephthalamide;
N-(2-Benzoyl-4-chloro-phenyl)-N′-[5-(3,5-dichloro-2-hydroxy-phenyl)-[1,3,4]thiadiazol-2-yl]-terephthalamide;
2-{4-[5-(3,5-Dichloro-2-hydroxy-phenyl)-[1,3,4]thiadiazol-2-ylcarbamoyl]-benzoylamino}-4,5-dimethoxy-benzoic acid methyl ester;
2-{4-[5-(3,5-Dichloro-2-hydroxy-phenyl)-[1,3,4]thiadiazol-2-ylcarbamoyl]-benzoylamino}-terephthalic acid dimethyl ester;
N-[2-(4-Chloro-benzoyl)-phenyl]-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(4-ethoxy-phenyl)-terephthalamide;
N-(3-Chloro-4-methoxy-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(2-methoxy-5-nitro-phenyl)-terephthalamide;
N-Biphenyl-4-yl-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(2-nitro-phenyl)-terephthalamide;
N-(3-Chloro-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(2,5-difluoro-phenyl)-terephthalamide;
N-[5-(3,5-Dichloro-2-hydroxy-phenyl)-[1,3,4]thiadiazol-2-yl]-N′-(3,5-difluoro-phenyl)-terephthalamide;
N-[5-(3,5-Dichloro-2-hydroxy-phenyl)-[1,3,4]thiadiazol-2-yl]-N′-(3,4-dichloro-phenyl)-terephthalamide;
N-(3-Cyano-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(4-hydroxy-phenyl)-terephthalamide;
2-{4-[5-(3,5-Dichloro-2-hydroxy-phenyl)-[1,3,4]thiadiazol-2-ylcarbamoyl]-benzoylamino}-4,5-dimethoxy-benzoic acid;
N-(4-Benzyloxy-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-methylsulfanyl-phenyl)-terephthalamide;
N-[5-(3,5-Dichloro-2-hydroxy-phenyl)-[1,3,4]thiadiazol-2-yl]-N′-(4-methylsulfanyl-phenyl)-terephthalamide;
N-[5-(3,5-Dichloro-2-hydroxy-phenyl)-[1,3,4]thiadiazol-2-yl]-N′-(9H-fluoren-1-yl)-terephthalamide;
N-(4-Carbamoyl-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-nitro-phenyl)-terephthalamide;
N-[4-(4-Bromo-1-methyl-1H-pyrazol-3-yl)-phenyl]-N′-[5-(3,5-dichloro-2-hydroxy-phenyl)-[1,3,4]thiadiazol-2-yl]-terephthalamide;
5-(4-{4-[5-(3,5-Dichloro-2-hydroxy-phenyl)-[1,3,4]thiadiazol-2-ylcarbamoyl]-benzoylamino}-phenyl)-2-methyl-furan-3-carboxylic acid ethyl ester;
N-[5-(3,5-Dichloro-2-hydroxy-phenyl)-[1,3,4]thiadiazol-2-yl]-N′-[4-(2-methyl-2H-pyrazol-3-yl)-phenyl]-terephthalamide;
N-[4-(5-tert-Butyl-[1,2,3]triazol-1-yl)-phenyl]-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-[4-(2H-tetrazol-5-yl)-phenyl]-terephthalamide;
N-[5-(3,5-Dichloro-2-hydroxy-phenyl)-[1,3,4]thiadiazol-2-yl]-N′-(4-imidazol-1-yl-phenyl)-terephthalamide;
N-[5-(3,5-Dichloro-2-hydroxy-phenyl)-[1,3,4]thiadiazol-2-yl]-N′-(4-[1,2,4]triazol-1-yl-phenyl)-terephthalamide;
Hydrochloride of N-[5-(3,5-Dichloro-2-hydroxy-phenyl)-[1,3,4]thiadiazol-2-yl]-N′-(4-[1,2,3]triazol-2-yl-phenyl)-terephthalamide;
Hydrochloride of N-[5-(3,5-Dichloro-2-hydroxy-phenyl)-[1,3,4]thiadiazol-2-yl]-N′-(4-[1,2,3]triazol-1-yl-phenyl)-terephthalamide;
N-[5-(3,5-Dichloro-2-hydroxy-phenyl)-[1,3,4]thiadiazol-2-yl]-N′-[4-(2-methyl-2H-tetrazol-5-yl)-phenyl]-terephthalamide;
N-(3-Chloro-4-[1,2,4]triazol-1-yl-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-methyl-4-[1,2,4]triazol-1-yl-phenyl)-terephthalamide;
3-Bromo-N-[5-(3,5-dichloro-2-hydroxy-phenyl)-[1,3,4]thiadiazol-2-yl]-benzamide;
4-Chloro-N-[5-(3,5-dichloro-2-hydroxy-phenyl)-[1,3,4]thiadiazol-2-yl]-benzamide;
3,4-Dichloro-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-3-iodo-benzamide;
N-[5-(3,5-Dichloro-2-hydroxy-phenyl)-[1,3,4]thiadiazol-2-yl]-4-methyl-3-nitro-benzamide′
Benzo[1,3]dioxole-5-carboxylic acid [5-(3,5-dichloro-2-hydroxy-phenyl)-[1,3,4]thiadiazol-2-yl]-amide;
3-Benzoyl-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-4-pyrrol-1-yl-benzamide;
3-Bromo-N-[5-(3,5-dichloro-2-hydroxy-phenyl)-[1,3,4]thiadiazol-2-yl]-4-methyl-benzamide;
4-Bromo-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-3,4-diethoxy-benzamide;
3-Bromo-N-[5-(3,5-dichloro-2-hydroxy-phenyl)-[1,3,4]thiadiazol-2-yl]-4-methoxy-benzamide;
3-Chloro-N-[5-(3,5-dichloro-2-hydroxy-phenyl)-[1,3,4]thiadiazol-2-yl]-4-methoxy-benzamide;
N-[5-(3,5-Dichloro-2-hydroxy-phenyl)-[1,3,4]thiadiazol-2-yl]-3-fluoro-4-methyl-benzamide;
N-[5-(3,5-Dichloro-2-hydroxy-phenyl)-[1,3,4]thiadiazol-2-yl]-4-iodo-benzamide;
4-Chloro-N-[5-(3,5-dichloro-2-hydroxy-phenyl)-[1,3,4]thiadiazol-2-yl]-3-methyl-benzamide;
4-Bromo-N-[5-(3,5-dichloro-2-hydroxy-phenyl)-[1,3,4]thiadiazol-2-yl]-3-methyl-benzamide;
N-[5-(3,5-Dichloro-2-hydroxy-phenyl)-[1,3,4]thiadiazol-2-yl]-4-propyl-benzamide;
N-[5-(3,5-Dichloro-2-hydroxy-phenyl)-[1,3,4]thiadiazol-2-yl]-4-ethoxy-benzamide;
N-[5-(3,5-Dichloro-2-hydroxy-phenyl)-[1,3,4]thiadiazol-2-yl]-4-methoxy-3-nitro-benzamide;
{4-[5-(3,5-Dichloro-2-hydroxy-phenyl)-[1,3,4]thiadiazol-2-ylcarbamoyl]-phenyl}-carbamic acid tert-butyl ester;
4-Butoxy-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-3,5-dihydroxy-benzamide;
Pyridine-2,5-dicarboxylic acid 5-{[5-(3,5-dichloro-2-hydroxy-phenyl)-[1,3,4]thiadiazol-2-yl]-amide}2-phenylamide;
Pyridine-2,5-dicarboxylic acid 2-{[5-(3,5-dichloro-2-hydroxy-phenyl)-[1,3,4]thiadiazol-2-yl]-amide}5-phenylamide;
4-Benzoylamino-N-[5-(3,5-dichloro-2-hydroxy-phenyl)-[1,3,4]thiadiazol-2-yl]-benzamide; or
(ii) a pharmaceutically acceptable salt, prodrug, stereoisomer or solvate of (i) thereof.

### Methods of Preparation

The compounds of the present invention may be synthesized by many methods available to those skilled in the art of organic chemistry. General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Additionally, the various steps in the synthesis may be performed in an alternate sequence in order to give the desired compound or compounds. One of skill understand that while the schemes depicted below illustrate compounds where A=phenyl, that such procedures are generally applicable to intermediates and final compounds where A is aryl or heteroaryl.

Compounds of the invention may be prepared by according to Scheme 1. The desired benzoic acid (a) is converted to the corresponding benzoyl chloride (b) using typical reagents, i.e. thionyl chloride or oxalyl chloride in low-boiling solvents such as methylene chloride or ethyl acetate in the presence of DMF. The benzoyl chloride (b) is then condensed with thiosemicarbazide in, e.g. DMF. The intermediate (c) is then cyclized in a dehydrating medium such as toluene/methanesulfonic acid mixture or toluene/paratoluenesulfonic acid or trifluoroacetic acid to afford the corresponding aminothiadiazole (d). The aminothiadiazole (d)is then condensed with R₁₃COOH to give the desired amide thiadiazole (e) using typical coupling reagents such as EDCI/HOBt or DCC/HOBt in the presence of a base such as triethylamine or DIEA.

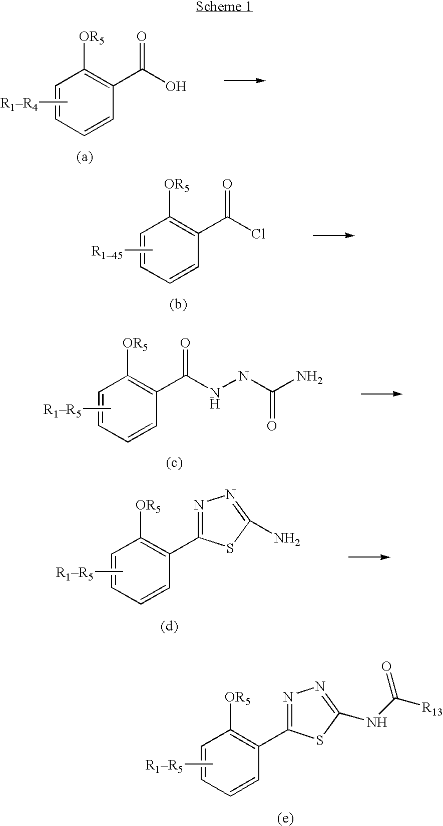

Alternatively, compounds of the present invention can be prepared as depicted in the following Scheme 2. Aminothiadiazole (d) is condensed with terephthaloyl dichloride in typical organic solvents such as THF, acetone or mixtures therof. The resulting intermediate (f) is condensed with the desired amine (NHR₁₅R₁₆) in the presence of a base such a pyridine, in typical organic solvent such as DMF, THF, or a mixture thereof, to afford the desired amide thiadiazole (g).

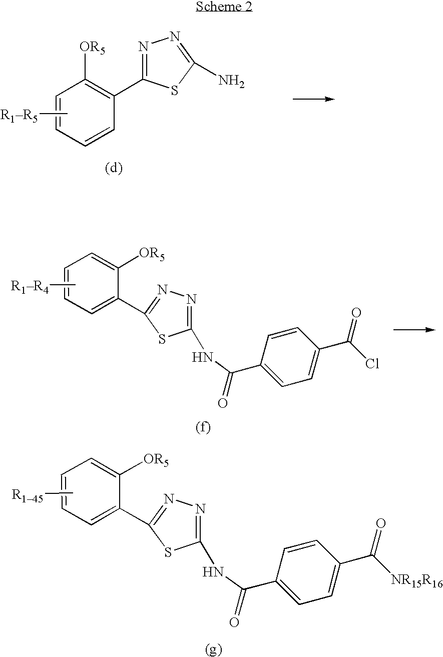

Alternatively, compounds of the present invention can be prepared as depicted in the following Scheme 3. The desired aminothiadiazole (d) is condensed with monomethylterephthalate using typical coupling reagents such as EDCI/HOBt or DCC/HOBt in the presence of a base such as triethylamine or DIEA in an organic solvent such as DMF. The obtained intermediate (h) may then be condensed with an amine (NHR₁₅R₁₆) in the presence of a base such as triethylamine or DIEA in appropriate organic solvents such as methylene chloride, or ethyl acetate to afford the final amide thiadiazole (j)

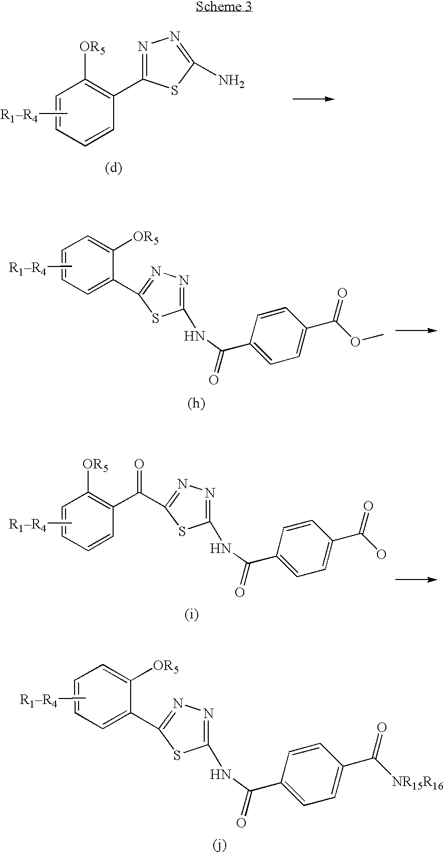

### Utility

The compounds of this invention are inhibitors of PAI-1 and are useful for the treatment or prevention of thromboembolic disorders in mammals (i.e., PAI-1 associated disorders). In general, a thromboembolic disorder is a circulatory disease caused by blood clots (i.e., diseases involving fibrin formation, platelet activation, and/or platelet aggregation). The term “thromboembolic disorders” as used herein includes arterial cardiovascular thromboembolic disorders, venous cardiovascular thromboembolic disorders, thromboembolic disorders in the chambers of the heart and fibrin accumulation in the microcirculation as occurs in disseminated intravascular coagulation. The term “thromboembolic disorders” as used herein also includes specific disorders selected from, but not limited to, unstable angina or other acute coronary syndromes, first or recurrent myocardial infarction, ischemic sudden death, transient ischemic attack, stroke, atherosclerosis, peripheral occlusive arterial disease, venous thrombosis, deep vein thrombosis, thrombophiebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g., after a bypass) and reocclusion (e.g., during or after percutaneous transluminal coronary angioplasty). The thromboembolic disorders may result from conditions including but not limited to atherosclerosis, surgery or surgical complications, prolonged immobilization, arterial fibrillation, congenital thrombophilia, cancer, diabetes, effects of medications or hormones, and complications of pregnancy. The anti-thrombotic effect of compounds of the present invention is believed to be due to inhibition of PAI-1.

The effectiveness of compounds of the present invention as inhibitors of PAI-1, can be determined using a relevant purified serine protease, respectively, and an appropriate synthetic substrate. The rate of hydrolysis of the chromogenic or fluorogenic substrate by the relevant serine protease was measured both in the absence and presence of compounds of the present invention. Hydrolysis of the substrate resulted in the release of pNA (para nitroaniline), which was monitored spectrophotometrically by measuring the increase in absorbance at 405 nm. A decrease in the rate of absorbance or fluorescence change in the presence of inhibitor is indicative of enzyme inhibition. Such methods are known to one skilled in the art. The results of this assay are expressed as the inhibitory constant, Kᵢ.

Chromogenic assays for PAI-1 inhibitors were conducted using either tPA or Urokinase as a “substrate” for PAI-1 at a final assay volume of 200 uL with 1% DMSO, a final concentration of either 2 nM t-PA ((Alteplase, 1 mg/ml reconstituted from InTime 1 kit #193) or 10 units/mL urokinase (Abbokinase, Abbott, Chicago, Ill.). An amount of human PAI-1 (Molecular Innovations, Inc, PAI-A) sufficient to neutralize t-PA or urokinase was used. The chromogenic assay was started by adding either spectrozyme tPA substrate (American Diagnotica, Inc. CT, #444L) or S2444 from chromogenix as the substrate for urokinase. The inhibition of PAI-1 activity was determined by comparing the rate of the reactions in the absence of inhibitor, but in the presence of PAI-1. Compounds tested in the chromogenic assay are considered to be active if they exhibit Kᵢ's of equal to or less than 30 μM.

Human fibrinolysis assays were conducted by diluting the compounds of interest into assay buffer consisting of 50 mM potassium phosphate, pH 7.4 and 0.05% BSA. Human PAI-1 (Molecular Innovations Inc., Michigan, CAT #PAI-A), then tPA (Genetech, Calif.), human glu-plasminogen (Enzyme research laboratories (ERL), Indiana HPg2001), and plaminogen depleted human fibrinogen (ERL, HFN) were added sequentially, then followed by the addition of human thrombin (ERL, HT1002 ). Compounds tested in the fibrinolysis assay are considered to be active if they exhibit a Kᵢ of equal to or less than 30 μM.

Compounds of the present invention have demonstrated Kᵢ values of equal to or less than 30 μM, preferably less than 10 μM, more preferably less than 2 μM, and even more preferably less than 1.5 μM in at least one of the above assays, thereby confirming the utility of the compounds of the present invention as effective inhibitors of PAI-1 and useful for the prevention or treatment of thromboembolic disorders in mammals.

The antithrombotic effect of compounds of the present invention can be can be demonstrated using relevant in vivo thrombosis model including in a rat model of acute venous thrombosis where the thrombolytic state is induced by submaximal tPA.

In this model male SD rats (300-400 g) are anesthetized with 60 mg/kg, i.p. Na-pentobarbital. PE-205 tubing is inserted in the trachea to maintain airway patency. PE-50 catheters are inserted in both jugular veins to administer test articles and in a femoral vein to inject human recombinant tissue factor (hrTF). The vena cava is isolated by a midline abdominal incision and temperature maintained with heating lamps. A fixed stenosis is produced just distal to the renal veins by tying a ligature around a 26-gauge steel tubing that was laid alongside the vena cava segment and then removing the tubing. Thrombosis is induced with a 1.4 mL/kg infusion of hrTF (1/10 dilution RecombiPlasTin®,Ortho Diagnostics) given over 2 min. The vena cava thrombus was removed and weighed 20 min after the start of hrTF infusion.

Treatment protocol includes i.v. infusion of a PAI-1 inhibitor of the present invention followed, after 5 minutes, by an i.v. infusion of human recombinant tissue plasminogen activator (tPA, Activase, Genentech) at a submaximal dose of 10 μg/kg/min. The hrTF is administered 1 min into the tPA infusion. The PAI-1 inhibitors are administered as a loading i.v. injection plus sustaining i.v. infusion at appropriate dose levels (mg/kg+mg/kg/hr) e.g.:at 3+3 and 10+10, and 2+2 and 5+5. Both tPA and PAI-1 inhibitor infusions are maintained until thrombus removal. The administration of the compounds of the present invention to rats treated with the submaximal dose of tPA results in significantly reduced thrombus weight.

The compounds of formula I and salts thereof are inhibitors of PAI-1, a major regulatory component of the plasminogen-plasmin system. As such, compounds of the present invention are useful in the treatment, inhibition, prevention or prophylaxis in a mammal, preferably in a human, of processes involving the production and/or action of PAI-1. See e.g. Binder et al., News Physiol. Sci., 17: 56-61 (2002) and Tsikouris, J. et al., J. Clin. Pharmacol., 42:1187-1199 (2002).

Accordingly, the compounds of the present invention may also be used in treating conditions including, but not limited to, metabolic diseases correlated to triacylglycerol levels & insulin resistance such as diabetes mellitus (Type 1 & 2), hyperinsulinemia, hyperglycemia and hypertriglyciridemia; obesity, acute & chronic inflammatory lung disorder such as respiratory distress syndrome, asthma, COPD, idiopathic pulmonary fibrosis, hyperoxide lung injury and bronchopulmonary dysplasia; renal disorders such as nephritic syndrome and hemolytic uremic syndrome; and malignancies such as tumor cell invasion, metastasis and neovascularization.

The compounds of the invention are also useful for the treatment of blood and blood products used in dialysis, blood storage in the fluid phase, especially ex vivo platelet aggregation. The present compounds may also be added to human plasma during the analysis of blood chemistry in hospital settings to determine the fibrinolytic capacity thereof.

The compounds of the present invention may also be used to treat cancer including, but not limited to, breast and ovarian cancer, and as imaging agents for the identification of metastatic cancers.

The compounds of the invention may also be used in the treatment of Alzheimer's disease. This method may also be characterized as the inhibition of plasminogen activator by PAI-1 in a mammal, particularly a human, experiencing or subject to Alzheimer's disease. This method may also be characterized as a method of increasing or normalizing levels of plasmin concentration in a mammal, particularly those experiencing or subject to Alzheimer's disease.

The compounds of the invention may be used for the treatment of myelofibrosis with myeloid metaplasia by regulating stromal cell hyperplasia and increases in extracellular matrix proteins.

The compounds of the invention may also be used in conjunction with protease inhibitor-containing highly active antiretroviral therapy (HAART) for the treatment of diseases which orginate from fibrinolytic impairment and hyper-coagulability of HIV-1 infected patients receiving such therapy.

The compounds of the invention may be used for the treatment of diabetic nephropathy and renal dialysis associated with nephropathy.

The compounds of the invention may be used to treat polycystic ovary syndrome, organ transplant rejection, septic shock and vascular damage associated with infections, cancer, septicemia, obesity, insulin resistance, proliferative diseases such as psoriasis, improving coagulation homeostasis, cerebrovascular diseases, microvascular disease, hypertension, dementia, arthritis, asthma, heart failure, arrhythmia, angina, and as a hormone replacement agent, treating, preventing or reversing progression of atherosclerosis, Alzheimer's disease, osteoporosis, osteopenia; reducing inflammatory markers, reducing C-reactive protein, or preventing or treating low grade vascular inflammation, stroke, coronary heart disease, primary and secondary prevention of myocardial infarction, stable and unstable angina, primary prevention of coronary events, secondary prevention of cardiovascular events, peripheral vascular disease, peripheral arterial disease including peripheral arterial occlusion, acute vascular syndromes, reducing the risk of undergoing a myocardial revascularization procedure, microvascular diseases such as nephropathy, neuropathy, retinopathy and nephrotic syndrome, hypertension, Type I and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improving coagulation homeostasis, and/or improving endothelial function, and all forms of cerebrovascular diseases.

The compounds of the invention may be used for the topical applications in wound healing for prevention of scarring.

The compounds of the present invention can be administered alone or in combination with one or more additional therapeutic agents. These include anti-coagulant or coagulation inhibitory agents, anti-platelet or platelet inhibitory agents, thrombin inhibitors, or thrombolytic or fibrinolytic agents.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e. prevent, inhibit or ameliorate) the thromboembolic disease condition or treat the progression of the disease in a host.

The compounds of the invention are preferably administered alone to a mammal in a therapeutically effective amount. However, the compounds of the invention can also be administered in combination with an additional therapeutic agent, as define below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased antiviral effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

Compounds which can be administered in combination with the compounds of the present invention include, but are not limited to, anticoagulants, anti-thrombin agents, anti-platelet agents, fibrinolytics, hypolipidemic agents, antihypertensive agents, and anti-ischemic agents.

Anticoagulant agents (or coagulation inhibitory agents) that may be used in combination with the compounds of this invention include warfarin, heparin, low molecular weight heparin (for example LOVANOX™), as well as other factor VIIa, VIIIa, IXa, Xa, XIa, prothrombin, TAFI, and fibrinogen inhibitors known in the art. The term anti-platelet agents (or platelet inhibitory agents), as used herein, denotes agents that inhibit platelet function such as by inhibiting the aggregation, adhesion or granular secretion of platelets. Such agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, and piroxicam, including pharmaceutically acceptable salts, hydrates or prodrugs thereof. Of the NSAIDS, aspirin (acetylsalicylic acid or ASA), and piroxicam are preferred. Other suitable anti-platelet agents include clopidrogel and ticlopidine, including pharmaceutically acceptable salts, hydrates or prodrugs thereof. Ticlopidine is also a preferred compound since it is known to be gentle on the gastro-intestinal tract in use. Still other suitable platelet inhibitory agents include IIb/IIIa antagonists, thromboxane-A2-receptor antagonists and thromboxane-A2-synthetase inhibitors, prostacyclin mimetics, phosphodiesterase (PDE) inhibitors, such as dipyridamole or cilostazol, serotonin-2-receptor antagonists, and P2Y₁ and P2Y₁₂ receptor antagonists, as well as pharmaceutically acceptable salts, hydrates or prodrugs thereof. Preferred P2Y₁₂ receptor antagonists include ticlopidine and clopidogrel, including pharmaceutically acceptable salts, hydrates or prodrugs thereof. Clopidogrel is an even more preferred agent. Ticlopidine and clopidogrel are also preferred compounds since they are known to be gentle on the gastro-intestinal tract in use.

The term thrombolytics (or fibrinolytic) agents (or thrombolytics or fibrinolytics), as used herein, denotes agents that lyse blood clots (thrombi). Such agents include tissue plasminogen activator (TPA), anistreplase, urokinase, streptokinase, PAI-1 inhibitors, and inhibitors of alpha-2-antiplasmin, including pharmaceutically acceptable salts, hydrates or prodrugs thereof. The term anistreplase, as used herein, refers to anisoylated plasminogen streptokinase activator complex, as described, for example, in European Patent Application No. 028,489, the disclosure of which is hereby incorporated herein by reference herein. The term urokinase, as used herein, is intended to denote both dual and single chain urokinase, the latter also being referred to herein as prourokinase.

The term hypolipidemic agents, as used herein, includes HMG-CoA reductase inhibitors (for example, pravastatin, simvastatin, atorvastatin, and the like) and microsomal triglyceride transport protein inhibitors.

The term antihypertensive agents, as used herein, includes angiotensin-converting enzyme inhibitors (for example captopril, lisinopril, or fosinopril), angiotensin-II receptor antagonists (for example irbestatin, losartan, or valsartan), ACE/NEP inhibitors (for example omapatrilat or gemopatrilat), diuretics (for example furosemide, chlorothiazide, or amiloride) and β-blockers (for example propanolol, nadolo, or carvedilol).

Administration of the compounds of the present invention of the invention in combination with such additional therapeutic agent, may afford an efficacy advantage over the compounds and agents alone, and may do so while permitting the use of lower doses of each. A lower dosage minimizes the potential of side effects, thereby providing an increased margin of safety.

The compounds of the present invention are also useful as standard or reference compounds, for example as a quality standard or control, in tests or assays involving the inhibition of PAI-1. Such compounds may be provided in a commercial kit, for example, for use in pharmaceutical research involving PAI-1. For example, a compound of the present invention could be used as a reference in an assay to compare its known activity to a compound with an unknown activity. This would ensure the experimentor that the assay was being performed properly and provide a basis for comparison, especially if the test compound was a derivative of the reference compound. When developing new assays or protocols, compounds according to the present invention could be used to test their effectiveness.

The compounds of the present invention may also be used in diagnostic assays involving PAI-1 tissue-type plasminogen activator (“tPA”) and urinary type plasminogen activator (“uPA”). For example, the presence of tPA and/or uPA in an unknown sample could be determined by addition of the relevant chromogenic substrate, for example S2444 for uPA and spectrozyme (American Diagnostics, Inc., CT #444L) for tPA, to a series of solutions containing test sample and optionally one of the compounds of the present invention. If production of pNA is observed in the solutions containing test sample together with exogenous PAI-1 and a compound of the present invention, then one would conclude that selective PAI-1 activity was absent, rather than, for example, PAI-2 or PAI-3 activity.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt or hydrate form thereof; and (c) a package insert stating that the pharmaceutical composition can be used for the treatment of a thromboembolic disorder (as defined previously). In another embodiment, the package insert states that the pharmaceutical composition can be used in combination (as defined previously) with a second therapeutic agent to treat a thromboembolic disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### Biological Assays

Chromogenic Assays for Inhibitors of PAI-1.

Chromogenic assays for PAI-1 inhibitors were conducted in 96 well plates (Costar 25381-054). Reactions were conducted using either tPA or Urokinase as a “substrate” for PAI-1. Reactions were set up such that each well contained a final volume of 200 uL with 1% DMSO, a final concentration of either 2 nM t-PA ((Alteplase, 1 mg/ml reconstituted from InTime 1 kit #193) or 10 units/mL urokinase (Abbokinase, Abbott, Chicago, Ill.). An amount of human PAI-1 (Molecular Innovations, Inc, PAI-A) sufficient to neutralize t-PA or urokinase was used. Each well contained 50 uL of buffer (50 mM Tris pH 8.3, 0.1 M NaCl containing 100 uL of Tween 80/L; buffer was filtered using a 0.2 um filter) and 2 uL of each compound of interest which had been diluted in 100% DMSO. Next, 50 uL of 10.4 nM PAI-1, in buffer, was added and the plate was vortexed for 1 min followed by a 30 minute incubation at room temperature. Fifty microliters of either 8 nM t-PA or 40 units/ml urokinase was added and the plate was vortexed for 1 minute. The chromogenic assay was started by adding either 50 uL of 1 mM spectrozyme tPA substrate (American Diagnotica, Inc. CT, #444L) or 50 uL of the 0.4 mM S2444 from chromogenix as the substrate for urokinase. The absorbance was measured at 405 nm over 15 mins using the kinetic mode of a spectramax 190 plate reader (Molecular Devices). The inhibition of PAI-1 activity was determined by comparing the rate of the reactions in the absence of inhibitor, but in the presence of PAI-1.

### Human Fibrinolysis Assay Protocol

Human fibrinolysis assays were conducted in 96 well plates (Costar 25381-054). Stock solutions were diluted into assay buffer consisting of 50 mM potassium phosphate, pH 7.4 and 0.05% BSA. Compounds to be tested were serially diluted in 100% DMSO. Each well contained 50 uL assay buffer and 2 uL of a given concentration of the compound of interest. Fifty microliters of a 2 nM human PAI-1 (Molecular Innovations Inc., Michigan, CAT #PAI-A) was added to each well and the plate was incubated at room temperature for 5 minutes. Next, 50 uL of each of the following solutions was added to each well and mixed for one minute: 0.42 nM tPA (Genetech, Calif.), 800 nM human glu-plasminogen (Enzyme research laboratories (ERL), Indiana HPg2001), 2 mg/ml plaminogen depleted human fibrinogen (ERL, HFN). Finally, the reaction was initiated by the addition of 50 uL of 14 nM human thrombin (ERL, HT1002). The time course of the reaction was followed by measuring the absorbance at 405 nm using a spectramax plate reader in kinetic mode. The time course of the reaction was observed for 4 hours. The extended time course was used to monitor the shape of the reaction curve. The percent inhibition of the fibrinolysis was determined by comparing the absorbance values at the 4 hour time point for wells containing compounds and control wells which contained no compound.

### EXAMPLES

The following Examples illustrate embodiments of the inventive compounds and starting materials, and are not intended to limit the scope of the claims. For ease of reference, the following abbreviations are used herein:

Abbreviations

CH₃CN=acetonitrile
DCC=dicyclohexylcarbodiimide
DCE=dichloroethane
DCM=dichloromethane
DMAP=4-dimethylaminopyridine
DIPEA or DIEA=N,N-diisopropylethylamine
DME=1,2-dimethoxyethane
DMF=dimethyl formamide
EDCI=1-3-dimethylaminopropyl)-3-ethylcarbodiimide
Et₂O=diethyl ether
HOBT=1-hydroxybenzotriazole
EtOAc=ethyl acetate
HCl=hydrochloric acid
KOH=potassium hydroxide
K₂CO₃=potassium carbonate
LiAlH₄=lithium aluminum hydride
MeCN=acetonitrile
MeOH=methanol
MgSO₄=magnesium sulfate
NaH=sodium hydride
NaOH=sodium hydroxide
NMP=1-methyl-2-pyrrolidinone
SOCl₂=thionyl chloride
TEA=triethylamine
bp=boiling point
g=gram(s)
mg=milligram(s)
ml=milliliter
μl=microliter
l=liter
mmol=millimole
μmol=micromole
mol=mole
mp=melting point
RT=room temperature
Rt=retention time

NMR (Nuclear Magnetic resonnance was performed on a Brucker 200 spectrometer (s=singulet, d=doublet, t=triplet, dd=doublet of doublet, q=quadruplet, m=multiplet)

Elementary analysis were carried on a Carlo-Erba Mod 106 elementary analyzer

### Preparation 1

### 3,5-Dichloro-2-hydroxy-benzoyl chloride

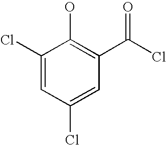

25 g of 3,5-dichlorosalicylic acid were suspended in 150 ml dichloromethane and 30 ml of thionyl chloride and 0.5 ml of DMF were added. The mixture was refluxed until a clear solution was obtained and the solvent evaporated, the temperature being maintained below 40° C., to yield a product used as such in Preparation 2.

¹H NMR (DMSO-d6): 9.92 (1H,s), 7.98 (1H, d, J=2 Hz), 7.68 (1H, d, J=2 Hz)

### Preparation 2

### 2-(5-Amino-[1,3,4]thiadiazol-2-yl)-4,6-dichloro-phenol

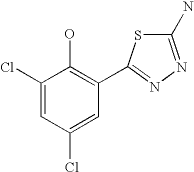

The product of Preparation 1 was dissolved in 100 ml THF, and 35 g of thiosemicarbazide dissolved in 120 ml DMF were added drop by drop. After 2 hours, the solvent was evaporated, the residue washed by 10% HCl water, filtered and dissolved in 250 ml toluene. 17 ml of methane sulfonic acid were added and the mixture was refluxed for 4 hours. After return to RT, the obtained precipitate was filtered, washed with acetone and with ammonia. The obtained precipitate was washed by hot acetone to give 16 g of a product melting at 268° C.

¹H NMR (DMSO-d6): 7.78 (1H, d, J=2 Hz), 7.75 (2H, s), 7.58 (1H, d, J=2 Hz)

### Preparation 3

### 4-[5-(3,5-Dichloro-2-hydroxy-phenyl)-[1,3,4]thiadiazol-2-ylcarbamoyl]-benzoyl chloride

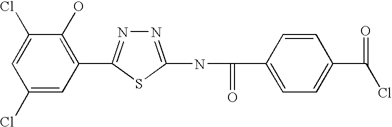

2 g of the product of Preparation 2 were dissolved in 60 ml THF and were added dropwise to 10 g of terephthaloyl dichloride dissolved in 150 ml of acetone. The mixture was stirred 30 mn at RT and the obtained precipitate was filtrated and washed with acetone and heated in 100 ml THF. The precipitate obtained in hot THF was discarded and THF was evaporated. The residue was triturated in acetone and filtrated to give 420 mg of a white product.

¹H NMR (DMSO-d6): 8.22 (2H, d), 8.02 (2H, d), 7.85 (1H, d), 7.65 (1H,d)

### Example 1

### N-[5-(3,5-Dichloro-2-hydroxy-phenyl)-[1,3,4]thiadiazol-2-yl]-terephthalamic acid methyl ester

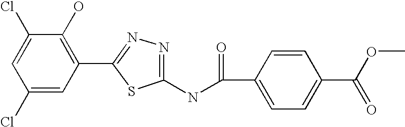

### 1A: 3,5-Dichloro-2-hydroxy-benzoyl chloride

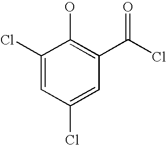

25 g of 3,5-dichlorosalicylic acid were suspended in 150 ml dichloromethane and 30 ml of thionyl chloride and 0.5 ml of DMF were added. The mixture was refluxed until a clear solution was obtained and the solvent evaporated, the temperature being maintained below 40° C., to yield compound 1A.

¹H NMR (DMSO-d6): 9.92 (1H,s), 7.98 (1H, d, J=2 Hz), 7.68 (1H, d, J=2 Hz)

### 1B: 2-(5-Amino-[1,3,4]thiadiazol-2-yl)-4,6-dichloro-phenol

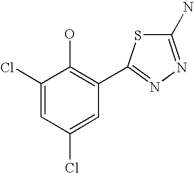

The product, 1A, was dissolved in 100 ml THF, and 35 g of thiosemicarbazide dissolved in 120 ml DMF were added drop by drop. After 2 hours, the solvent was evaporated, the residue washed by 10% HCl water, filtered and dissolved in 250 ml toluene. 17 ml of methane sulfonic acid were added and the mixture was refluxed for 4 hours. After return to RT, the obtained precipitate was filtered, washed with acetone and with ammonia. The obtained precipitate was washed by hot acetone to give 16 g of a product melting at 268° C.

¹H NMR (DMSO-d6): 7.78 (1H, d, J=2 Hz), 7.75 (2H, s), 7.58 (1H, d, J=2 Hz)

Title Compound

Product 1B (2.6 g) was dissolved in DMF and 1.8 g of monomethylterephthalate, 1.6 g of HOBT and 2.1 ml of DIEA were added. The mixture was stirred at RT for 30 mn and 2.3 g of EDCI were then added. The reaction mixture was heated at 65° C. for 2 hours, water was added and the obtained precipitate was washed by dilute HCl and acetone to give 2.3 g of a product melting over 300° C.

Microanalysis: theory(%): C: 48.1; H: 2.61; N: 9.90; obtained (%): C: 48.1; H: 2.85; N: 9.99

### Example 2

### N-[5-(3,5-Dichloro-2-hydroxy-phenyl)-[1,3,4]thiadiazol-2-yl]-terephthalamic acid

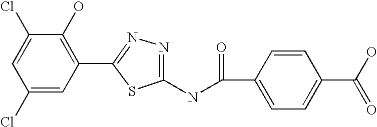

1.8 g of the product of Example 1 was dissolved in THF and 15 ml of NaOH 1N were added. The reaction mixture was heated at 45° C. for 2 hours and aqueous HCl was added. The obtained precipitate was washed with methanol and acetone to give 1.6 g of a product melting over 300° C.

Microanalysis: theory(%, 0.5H₂0): C, 45.8; H, 2.40; N, 10.0; obtained (%): C, 45.61 H, 2.43; N, 9.70.

### Example 3

### N-[5-(3,5-Dichloro-2-hydroxy-phenyl)-[1,3,4]thiadiazol-2-yl]-N′-phenyl-terephthalamide

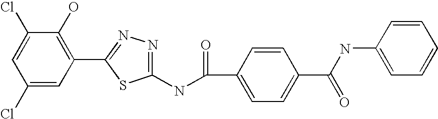

0.3 g of the product of Example 2 were dissolved in DMF and 0.75 g of aniline, 0.13 g of HOBT and 0.2 ml of DIEA were added. The mixture was stirred at RT for 30 mn and 0.12 g of EDCI were then added. The reaction mixture was heated at 60° C. for 2 hours, water was added and the obtained precipitate was washed by dilute HCl and acetone to give 0.3 g of a product melting at 342° C.

Microanalysis: theory(%): C, 54.4; H, 2.91; N, 11.5; obtained (%): C, 54.1; H, 2.90; N, 11.6.

### Examples 4 and 5

Example 4 and 5 were obtained from the product of Example 2 and corresponding anilines according to the procedure of Example 3

### Example 4

### N-[5-(3,5-Dichloro-2-hydroxy-phenyl)-[1,3,4]thiadiazol-2-yl]-N′-(4-phenoxy-phenyl)-terephthalamide

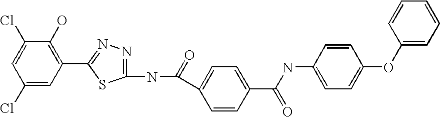

Microanalysis: theory(%): C, 58.2; H, 3.14; N, 9.70; obtained (%): C, 57.8; H, 3.30; N, 9.74.

### Example 5

### 4-(4-{4-[5-(3,5-Dichloro-2-hydroxy-phenyl)-[1,3,4]thiadiazol-2-ylcarbamoyl]-benzoylamino}-phenoxy)-benzoic acid methyl ester

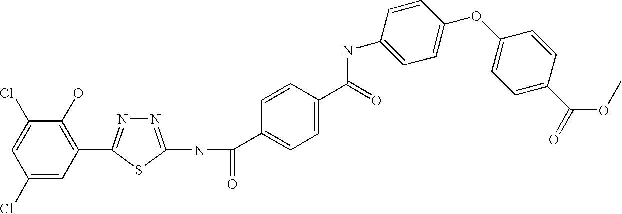

mp: 331

Microanalysis: theory(%): C, 56.7; H, 3.17; N, 8.82; obtained (%): C, 56.5; H, 3.22; N, 8.64.

### Example 6

### 4-(4-{4-[5-(3,5-Dichloro-2-hydroxy-phenyl)-[1,3,4]thiadiazol-2-ylcarbamoyl]-benzoylamino}-phenoxy)-benzoic acid

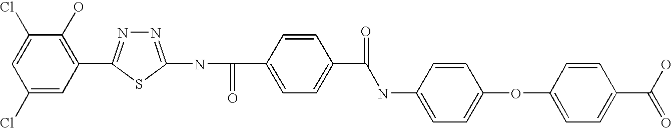

0.1 g of the product of Example 5 was dissolved in methanol and 1 ml of NaOH 1N were added. The reaction mixture was stirred at RT for 4 hours and aqueous HCl was added. The obtained precipitate was washed with methanol and acetone to give 0.035 g of a white product.

Microanalysis: theory(%, 2H₂0): C, 52.9; H, 3.37; N, 8.52; obtained (%): C, 52.9; H, 3.11; N, 8.25.

### Example 7

### 3-{4-[5-(3,5-Dichloro-2-hydroxy-phenyl)-[1,3,4]thiadiazol-2-ylcarbamoyl]-benzoylamino}-benzoic acid ethyl ester

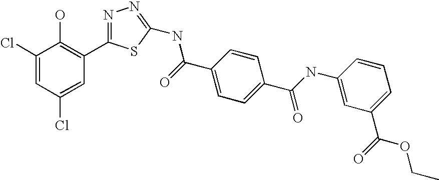

### 7A: 4-[5-(3,5-Dichloro-2-hydroxy-phenyl)-[1,3,4]thiadiazol-2-ylcarbamoyl]-benzoyl chloride

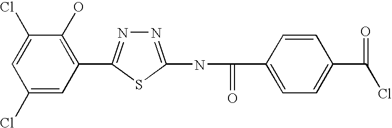

2 g of the product, 1B, was dissolved in 60 ml THF and added dropwise to 10 g of terephthaloyl dichloride dissolved in 150 ml of acetone. The mixture was stirred 30 mn at RT and the obtained precipitate was filtrated and washed with acetone and heated in 100 ml THF. The precipitate obtained in hot THF was discarded and THF was evaporated. The residue was triturated in acetone and filtrated to give 420 mg of a white product.

¹H NMR (DMSO-d6): 8.22 (2H, d), 8.02 (2H, d), 7.85 (1H, d), 7.65 (1H,d)

Title Compound

A mixture of the product 7A (20 mg; 0.046 mmole) in DMF (1 ml), 3-ethoxycarbonyl aniline (0.12 mmole) in THF (1 ml) and pyridine (10 □1) was stirred at 55° C. during 20 hrs. The reaction mixture was then allowed to cool down to room temperature and HCl in isopropanol (5.5 M, 100 □1) and water (c.a. 1.5 ml) were added. The precipitate was filtered, washed with a 30:70 methanol/water solution and dried to yield the desired compound. The final product was characterized by LC Mass (LCMS conditions: LC Micromass platform (APCI+, DAD (210-400 nm)), Column: TSK gel Super ODS 4.6 mm ID×5 cm, Flow rate: 2.75 mL/min, Gradient: from 100% eluent A to 100% eluent B in 2 min., with a plateau with 100% eluent B during 1 min. Eluent A: H₂O (0.05% TFA), Eluent B: CH₃CN/H₂O/TFA (80/20/0.05)).

LCMS: Rt: 2.50 mn; M/z: 558; Purity: 91%

### Example 8 to 44

The products of Example 8 to 44 were obtained from the product of 7A using the procedure described for Example 7

### Example 8

### N-(4-Chloro-phenyl)-N′-[5-(3,5-dichloro-2-hydroxy-phenyl)-[1,3,4]thiadiazol-2-yl]-terephthalamide

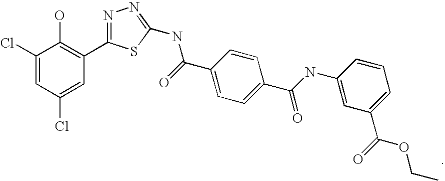

LCMS: Rt: 2.51 mn; M/z: 520; Purity: 83%

### Example 9

### N-[5-(3,5-Dichloro-2-hydroxy-phenyl)-[1,3,4]thiadiazol-2-yl]-N′-(3-fluoro-phenyl)-terephthalamide

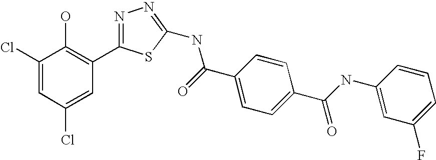

LCMS: Rt: 2.50 mn; M/z: 504; Purity: 86%

### Example 10

### N-[5-(3,5-Dichloro-2-hydroxy-phenyl)-[1,3,4]thiadiazol-2-yl]-N′-(4-fluoro-phenyl)-terephthalamide

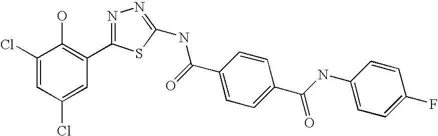

LCMS: Rt: 2.42 mn; M/z: 504; Purity: 82%

### Example 11

### N-[5-(3,5-Dichloro-2-hydroxy-phenyl)-[1,3,4]thiadiazol-2-yl]-N′-(9-ethyl-9H-carbazol-3-yl)-terephthalamide

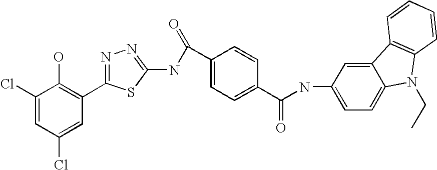

LCMS: Rt: 2.63 mn; M/z: 603; Purity: 90%

### Example 12

### N-[5-(3,5-Dichloro-2-hydroxy-phenyl)-[1,3,4]thiadiazol-2-yl]-N′-(4-pentyloxy-phenyl)-terephthalamide

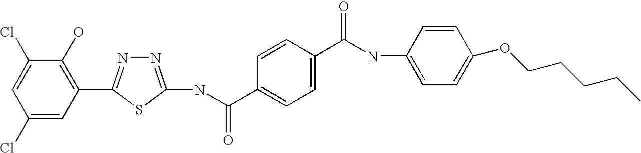

LCMS: Rt: 2.74 mn; M/z: 572; Purity: 99%

### Example 13

### N-[5-(3,5-Dichloro-2-hydroxy-phenyl)-[1,3,4thiadiazol-2-yl]-N′-(4-phenylamino-phenyl)-terephthalamide

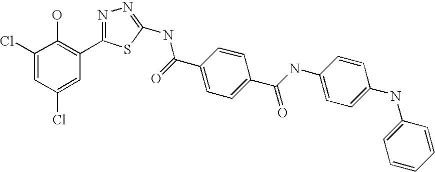

LCMS: Rt: 2.62 mn; M/z: 577; Purity: 83%

### Example 14

### 2-{4-[5-(3,5-Dichloro-2-hydroxy-phenyl)-[1,3,4]thiadiazol-2-ylcarbamoyl]-benzoylamino}-benzoic acid methyl ester

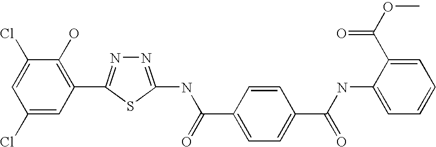

LCMS: Rt: 2.39 mn; M/z: 544; Purity: 100%

### Example 15

### N-[5-(3,5-Dichloro-2-hydroxy-phenyl)-[1,3,4]thiadiazol-2-yl]-N′-p-tolyl-terephthalamide

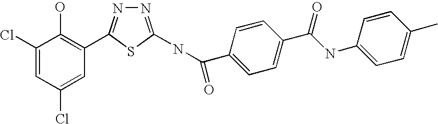

LCMS: Rt: 2.44 mn; M/z: 500; Purity: 81%

### Example 16

### N-(2-Benzoyl-4-chloro-phenyl)-N′-[5-(3,5-dichloro-2-hydroxy-phenyl)-[1,3,4]thiadiazol-2-yl]-terephthalamide

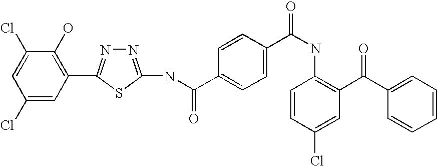

LCMS: Rt: 2.65 mn; M/z: 625; Purity: 86%

### Example 17

### 2-{4-[5-(3,5-Dichloro-2-hydroxy-phenyl)-[1,3,4]thiadiazol-2-ylcarbamoyl]-benzoylamino}-4,5-dimethoxy-benzoic acid methyl ester

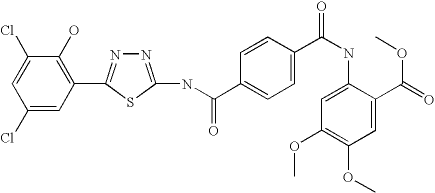

LCMS: Rt: 2.55 mn; M/z: 604; Purity: 90%

### Example 18

### 2-{4-[5-(3,5-Dichloro-2-hydroxy-phenyl)-[1,3,4]thiadiazol-2-ylcarbamoyl]-benzoylamino}-terephthalic acid dimethyl ester

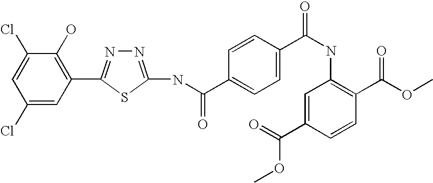

LCMS: Rt: 2.52 mn; M/z: 602; Purity: 92%

### Example 19

### N-[2-(4-Chloro-benzoyl)-phenyl]-N′-[5-(3,5-dichloro-2-hydroxy-phenyl)-[1,3,4]thiadiazol-2-yl]-terephthalamide

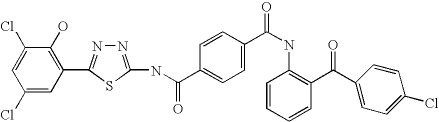

LCMS: Rt: 2.32 mn; M/z: 625; Purity: 100%

### Example 20

### N-[5-(3,5-Dichloro-2-hydroxy-phenyl)-[1,3,4]thiadiazol-2-yl]-N′-(4-ethoxy-phenyl)-terephthalamide

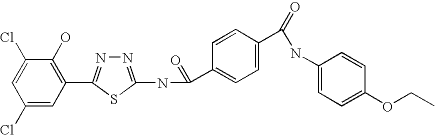

LCMS: Rt: 2.53 mn; M/z: 530; Purity: 88%

### Example 21

### N-(3-Chloro-4-methoxy-phenyl)-N′-[5-(3,5-dichloro-2-hydroxy-phenyl)-[1,3,4]thiadiazol-2-yl]-terephthalamide

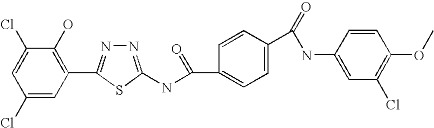

LCMS: Rt: 2.44 mn; M/z: 551; Purity: 78%

### Example 22

### N-[5-(3,5-Dichloro-2-hydroxy-phenyl)-[1,3,4]thiadiazol-2-yl]-N′-(2-methoxy-5-nitro-phenyl)-terephthalamide

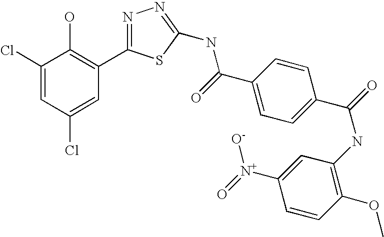

LCMS: Rt: 2.54 mn; M/z: 561; Purity: 80%

### Example 23

### N-Biphenyl-4-yl-N′-[5-(3,5-dichloro-2-hydroxy-phenyl)-[1,3,4]thiadiazol-2-yl]-terephthalamide

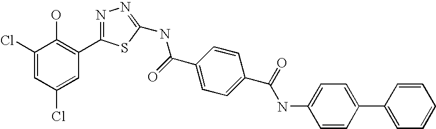

LCMS: Rt: 2.42 mn; M/z: 562; Purity: 78%

### Example 24

### N-[5-(3,5-Dichloro-2-hydroxy-phenyl)-[1,3,4]thiadiazol-2-yl]-N′-(2-nitro-phenyl)-terephthalamide

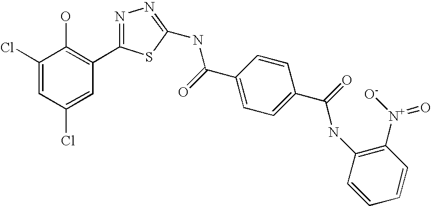

LCMS: Rt: 2.54 mn; M/z: 531; Purity: 100%

### Example 25

### N-(3-Chloro-phenyl)-N′-[5-(3,5-dichloro-2-hydroxy-phenyl)-[1,3,4]thiadiazol-2-yl]-terephthalamide

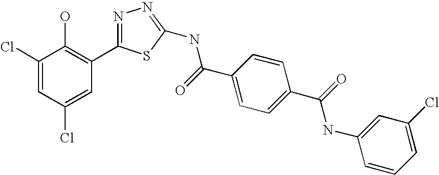

LCMS: Rt: 2.52 mn; M/z: 521; Purity: 98%

### Example 26

### N-[5-(3,5-Dichloro-2-hydroxy-phenyl)-[1,3,4]thiadiazol-2-yl]-N′-(2,5-difluoro-phenyl)-terephthalamide

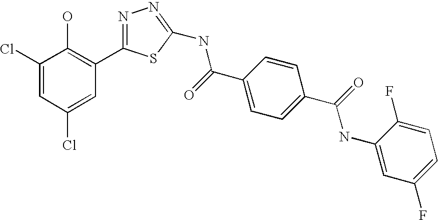

LCMS: Rt: 2.55 mn; M/z: 521; Purity: 100%

### Example 27

### N-[5-(3,5-Dichloro-2-hydroxy-phenyl)-[1,3,4]thiadiazol-2-yl]-N′-(3,5-difluoro-phenyl)-terephthalamide

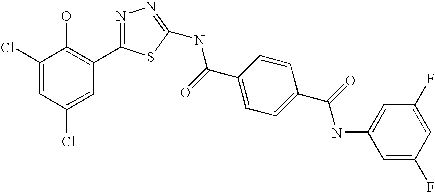

LCMS: Rt: 2.55 mn; M/z: 521; Purity: 89%

### Example 28

### N-[5-(3,5-Dichloro-2-hydroxy-phenyl)-[1,3,4]thiadiazol-2-yl]-N′-(3,4-dichloro-phenyl)-terephthalamide

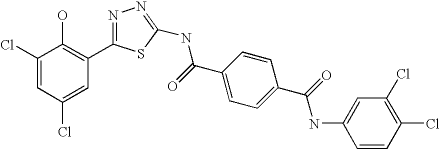

LCMS: Rt: 2.69 mn; M/z: 555; Purity: 97%

### Example 29

### N-(3-Cyano-phenyl)-N′-[5-(3,5-dichloro-2-hydroxy-phenyl)-[1,3,4]thiadiazol-2-yl]-terephthalamide

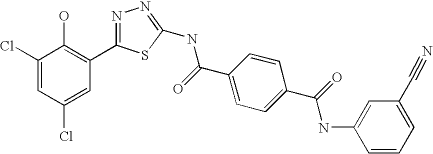

LCMS: Rt: 2.42 mn; M/z: 510; Purity: 98%

### Example 30

### N-[5-(3,5-Dichloro-2-hydroxy-phenyl)-[1,3,4]thiadiazol-2-yl]-N′-(4-hydroxy-phenyl)-terephthalamide

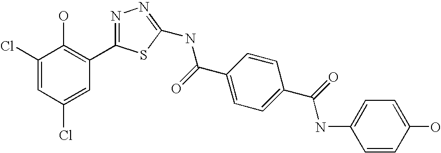

LCMS: Rt: 2.23 mn; M/z: 501; Purity: 100%

### Example 31

### 2-{4-[5-(3,5-Dichloro-2-hydroxy-phenyl)-[1,3,4]thiadiazol-2-ylcarbamoyl]-benzoylamino}-4,5-dimethoxy-benzoic acid

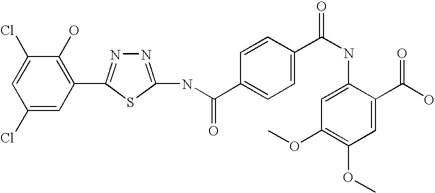

LCMS: Rt: 2.18 mn; M/z: 590; Purity: 95%

### Example 32

### N-(4-Benzyloxy-phenyl)-N′-[5-(3,5-dichloro-2-hydroxy-phenyl)-[1,3,4]thiadiazol-2-yl]-terephthalamide

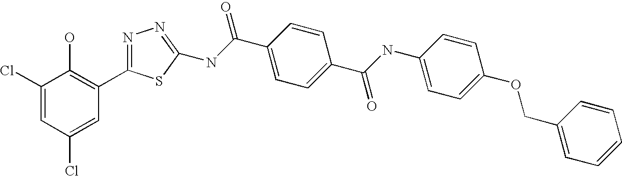

LCMS: Rt: 2.57 mn; M/z: 591; Purity: 85%

### Example 33

### N-[5-(3,5-Dichloro-2-hydroxy-phenyl)-[1,3,4]thiadiazol-2-yl]-N′-(3-methylsulfanyl-phenyl)-terephthalamide

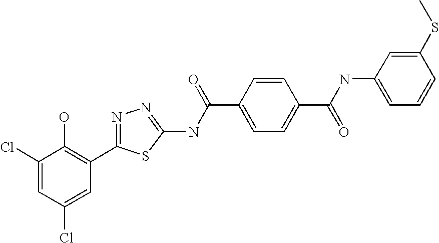

LCMS: Rt: 2.49 mn; M/z: 532; Purity: 92%

### Example 34

### N-[5-(3,5-Dichloro-2-hydroxy-phenyl)-[1,3,4]thiadiazol-2-yl]-N′-(4-methylsulfanyl-phenyl)-terephthalamide

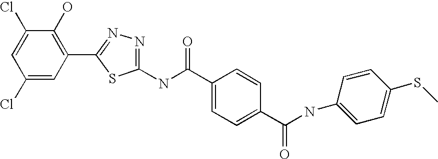

LCMS: Rt: 2.48 mn; M/z: 532; Purity: 91%

### Example 35

### N-[5-(3,5-Dichloro-2-hydroxy-phenyl)-[1,3,4]thiadiazol-2-yl]-N′-(9H-fluoren-1-yl)-terephthalamide

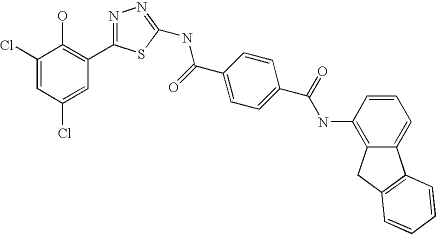

LCMS: Rt: 2.69 mn; M/z: 574; Purity: 95%

### Example 36

### N-(4-Carbamoyl-phenyl)-N′-[5-(3,5-dichloro-2-hydroxy-phenyl)-[1,3,4]thiadiazol-2-yl]-terephthalamide

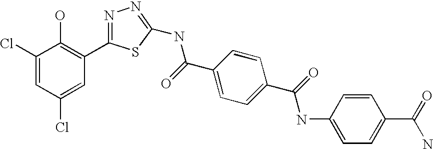

LCMS: Rt: 2.17 mn; M/z: 529; Purity: 90%

### Example 37

### N-[5-(3,5-Dichloro-2-hydroxy-phenyl)-[1,3,4]thiadiazol-2-yl]-N′-(3-nitro-phenyl)-terephthalamide

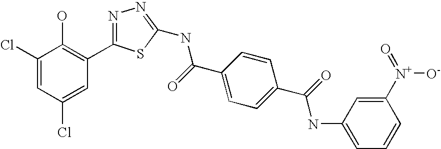

LCMS: Rt: 2.49 mn; M/z: 530; Purity: 91%

### Example 38

### N-[4-(4-Bromo-1-methyl-1H-pyrazol-3-yl)-phenyl]-N′-[5-(3,5-dichloro-2-hydroxy-phenyl)-[1,3,4]thiadiazol-2-yl]-terephthalamide

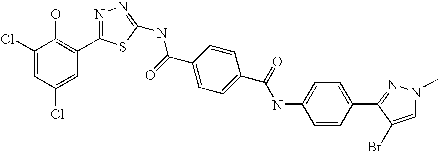

LCMS: Rt: 2.40 mn; M/z: 645; Purity: 96%

### Example 39

### N-[5-(3,5-Dichloro-2-hydroxy-phenyl)-[1,3,4]thiadiazol-2-yl]-N′-[4-(3-methyl-pyrazol-1-yl)-phenyl]-terephthalamide

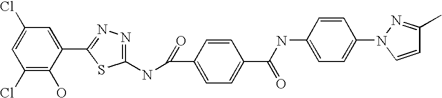

LCMS: Rt: 2.39 mn; M/z: 566; Purity: 100%

### Example 40

### 5-(4-{4-[5-(3,5-Dichloro-2-hydroxy-phenyl)-[1,3,4]thiadiazol-2-ylcarbamoyl]-benzoylamino}-phenyl)-2-methyl-furan-3-carboxylic acid ethyl ester

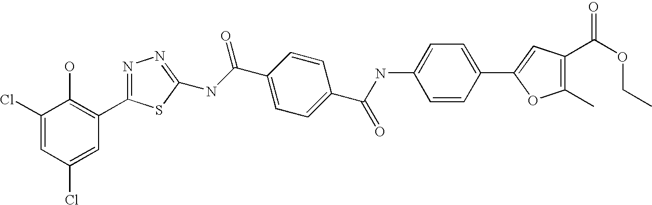

LCMS: Rt: 2.68 mn; M/z: 638; Purity: 90%

### Example 41

### N-[5-(3,5-Dichloro-2-hydroxy-phenyl)-[1,3,4]thiadiazol-2-yl]-N′-[4-(2-methyl-2H-pyrazol-3-yl)-phenyl]-terephthalamide

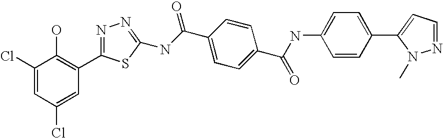

LCMS: Rt: 2.36 mn; M/z: 566; Purity: 83%

### Example 42

### N-[4-(5-tert-Butyl-[1,2,3]triazol-1-yl)-phenyl]-N′-[5-(3,5-dichloro-2-hydroxy-phenyl)-[1,3,4]thiadiazol-2-yl]-terephthalamide

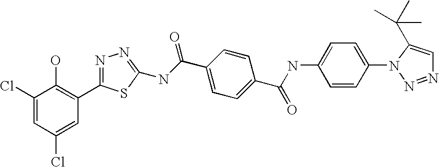

LCMS: Rt: 2.40 mn; M/z: 609; Purity: 93%

### Example 43

### N-[5-(3,5-Dichloro-2-hydroxy-phenyl)-[1,3,4]thiadiazol-2-yl]-N′-[4-(2H-tetrazol-5-yl)-phenyl]-terephthalamide

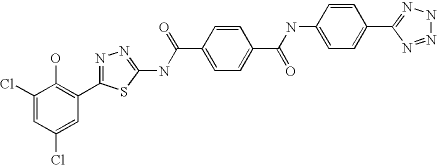

LCMS: Rt: 1.95 mn; M/z: 554; Purity: 100%

### Example 44

### N-[5-(3,5-Dichloro-2-hydroxy-phenyl)-[1,3,4]thiadiazol-2-yl]-N′-(4-imidazol-1-yl-phenyl)-terephthalamide

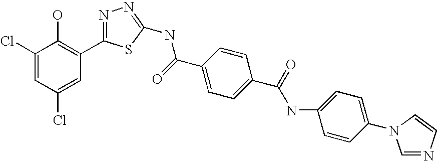

LCMS: Rt: 1.99 mn; M/z: 551; Purity: 89%

### Example 45

### N-[5-(3,5-Dichloro-2-hydroxy-phenyl)-[1,3,4]thiadiazol-2-yl]-N′-(4-[1,2,4]triazol-1-yl-phenyl)-terephthalamide

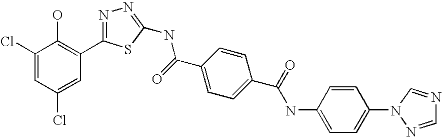

### 45A 1-(4-Nitro-phenyl)-1H-[1,2,4]triazole

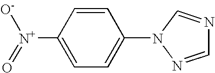

15 g of 1,2,4 triazole were dissolved in DMF and 8.8 g of NaH (60% in oil) were added. The mixture was stirred at RT for 6 hours and 28.2 g of 1 fluoro-4-nitrobenzene were added. After 6 hours at RT, water was added and the resulting precipitate was washed by ethanol to give 36 g of a product melting at 198° C. used as such in the next step.

### 45B: 4-[1,2,4]Triazol-1-yl-phenylamine

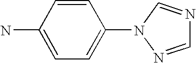

18 g of Intermediate 45A was dissolved in 300 ml of ethanol and 70 g of tin dichloride was added. The mixture was refluxed for 4 hours, ethanol was evaporated and dilute sodium hydroxide was added. After extraction with dichloromethane, the dichloromethane phase was dried over magnesium sulfate and evaporated. A powder was obtained, which was washed with pentane to give 12 g of a white solid.

¹H NMR (DMSO-d6): 9.00 (1H,s), 8.08 (1H,s), 7.44 (2H, d), 6.69 (2H,d), 5.55 (2H, broad signal).

Title Compound

300 mg of the product, 7A, was dissolved in THF and 0.15 g of Intermediate 45B and 0.1 ml of pyridine were added. The mixture was refluxed for 3 hours, then dilute HCl was added. A precipitate was obtained which was filtered and washed with acetone to give 200 mg of an off-white product.

Microanalysis: theory(%, 1 H₂0): C, 50.5; H, 3.00; N, 17.1; obtained (%): C, 50.5; H, 2.80; N, 16.9.

### Example 46

### Hydrochloride of N-[5-(3,5-Dichloro-2-hydroxy-phenyl)-[1,3,4]thiadiazol-2-yl]-N′-(4-[1,2,3]triazol-2-yl-phenyl)-terephthalamide

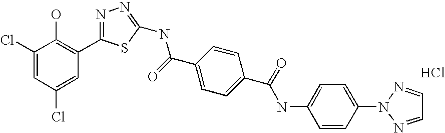

### 46A: 2-(4-Nitro-phenyl)-2H-[1,2,3]triazole

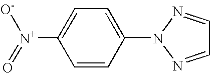

2.5 g of 1,2,3 triazole was dissolved in 40 ml DMF. 1.4 g of NaH (60% in oil) were added and the mixture was stirred at RT for 1 hour. 5 g of p-fluoronitrobenzne were added and the reaction mixture was stirred at RT for 4 hours. Water was added and a precipitate was isolated by filtration and dissolved in dichloromethane. A precipitate was discarded (fraction A, 0.95 g of a product melting at 210° C.) and the dichloromethane was evaporated to give a residue which was purified by chromatography on silica gel using dichloromethane as eluant to give 2.1 g of a product melting at 182° C.

¹H NMR (DMSO-d6): 8.45 (2H,d), 8.08 (2H,s), 8.35 (2H, s), 8.25 (2H,d)

### 46B: -[1,2,3]Triazol-2-yl-phenylamine

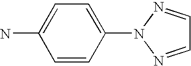

2.1 g of Intermediate 46A was dissolved in 50 ml of ethanol and 8 g of tin dichloride were added. The mixture was refluxed for 4 hours, ethanol was evaporated and dilute sodium hydroxide was added. The obtained precipitate ( 1.7 g) was filtered out and used as such in the following step without further purification

¹H NMR (DMSO-d6): 8.00 (2H,s), 7.75 (2H,d), 6.80 (2H, d), 5.45 (2H,broad signal)

Title Compound

200 mg of the product, 7A, was dissolved in THF and 0.15 g of Intermediate 46B and 0.1 ml of pyridine were added. The mixture was refluxed for 3 hours, then dilute HCl was added. A precipitate was obtained which was filtered and washed with acetone to give 120 mg of an off-white product.

Microanalysis: theory(%, 1 HCl): C, 48.9; H, 2.74; N, 16.6; obtained (%): C, 49.0; H, 2.80; N, 16.6.

### Example 47

### Hydrochloride of N-[5-(3,5-Dichloro-2-hydroxy-phenyl)-[1,3,4]thiadiazol-2-yl]-N′-(4-[1,2,3]triazol-1-yl-phenyl)-terephthalamide

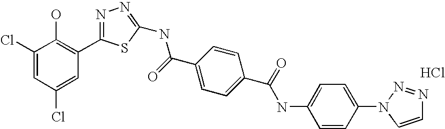

### 47A: 4-[1,2,3]Triazol-1-yl-phenylamine

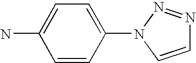

0.95 g of Fraction A, isolated in the preparation of Intermediate 46A was dissolved in 20 ml of ethanol and 4 g of tin dichloride was added. The mixture was refluxed for 4 hours, ethanol was evaporated and dilute sodium hydroxide was added. The mixture was extracted with dichloromethane and the dichloromethane evaporated to give 0.6 g of a product used as such in the following step without further purification.

¹H NMR (DMSO-d6): 8.51 (1H,s), 7.86 (1H,s), 7.65 (2H,d), 6.65 (2H, d), 5.50 (2H,broad signal)

Title Compound

200 mg of the product of 7A was dissolved in THF and 0.15 g of Intermediate 47A and 0.1 ml of pyridine were added. The mixture was refluxed for 3 hours, then dilute HCl was added. A precipitate was obtained which was filtered and washed with acetone to give 120 mg of an off-white product.

Microanalysis: theory(%, 1 HCl): C, 48.9; H, 2.74; N, 16.6; obtained (%): C, 49.0; H, 2.65; N, 16.7.

### Example 48

### N-[5-(3,5-Dichloro-2-hydroxy-phenyl)-[1,3,4]thiadiazol-2-yl]-N′-[4-(2-methyl-2H-tetrazol-5-yl)-phenyl]-terephthalamide

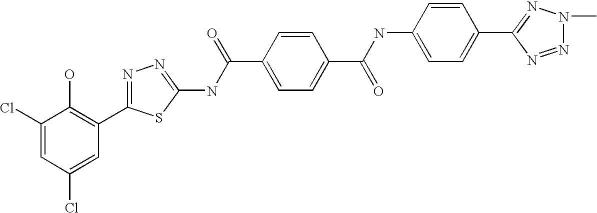

### 48A: 2-Methyl-5-(4-nitro-phenyl)-2H-tetrazole

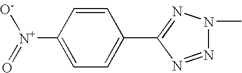

1.9 g of 5-(4-nitro-phenyl)-2H-tetrazole were dissolved in dichloromethane and 2 ml of diisopropylamine were added followed by 0.7 ml of methyl iodide. The mixture was stirred for 7 hours at RT and the organic phase was washed with water and evaporated. The residue was taken in ethanol and pentane was added until precipitation occurs. 0.95 g of a white precipitate was filtered out and used as such in the next step.

¹H NMR (DMSO-d6): 8.48 (2H,d), 8.25(2H,d), 4.50 (3H, s)

### 48B: 4-(2-Methyl-2H-tetrazol-5-yl)-phenylamine

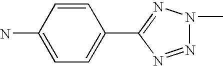

0.95 g of intermediate 48-1 were dissolved in ethanol, 5 g of stannyl dichloride were added and the mixture was refluxed for 4 hours. After return to RT, ethanol was evaporated, a dilute solution of NaOH was added and the mixture was extracted by dichioromethane. The dichloromethane phase was dried over magnesium sulfate and evaporated to give 0.65 g of a product used as such in the next step.

1H NMR (DMSO-d6): 7.70(2H,d), 6.65(2H,d), 7.65 (2H,d), 5.50 (2H,broad signal), 4.35 (3H, s)

Title Compound

320 mg of the product, 7A, was dissolved in THF and 0.600 g of Intermediate 48B and 0.1 ml of pyridine were added. The mixture was refluxed for 3 hours, then dilute HCl was added. A precipitate was obtained which was filtered and washed with acetone to give 125 mg of an off-white product.

Microanalysis: theory(%, 1 H₂0): C, 49.2; H, 3.10; N, 19.1; obtained (%): C, 50.4; H, 2.95; N, 19.0.

### Example 49

### N-(3-Chloro-4-[1,2,4]triazol-1-yl-phenyl)-N′-[5-(3,5-dichloro-2-hydroxy-phenyl)-[1,3,4]thiadiazol-2-yl]-terephthalamide

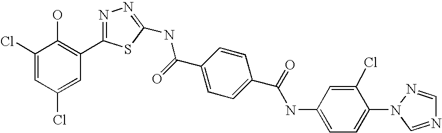

### 49A: 1-(2-Chloro-4-nitro-phenyl)-1H-[1,2,4]triazole

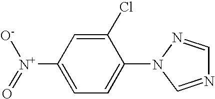

1.7 g of 1,2,4 triazole was dissolved in 70 ml of DMF and 1 g of NaH (60% in oil)was added. The mixture was stirred for 30 mn at RT and 1 equivalent 2-chloro-4-fluoro-nitrobenzene was added. The reaction mixture was stirred at RT for 1 day and water was added. The reaction mixture was extracted with dichloromethane, the dichloromethane phase was washed three times with water and evaporated. The residue was triturated in isopropyl ether to give 1.5 g of a yellow powder used as such in the next step.

¹H NMR (DMSO-d6): 9.15 (1H,s), 8.62 (1H,d), 8.46(1H,m), 8.35(1H, s), 7.95 (1H,d)

### 49B: 3-Chloro-4-[1,2,4]triazol-1-yl-phenylamine

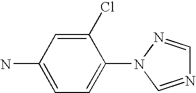

0.95 g of intermediate 49A+ were dissolved in ethanol, 3 g of stannyl dichloride were added and the mixture was refluxed for 4 hours. After return to RT, ethanol was evaporated, a dilute solution of NaOH was added and the mixture was extracted by dichloromethane. The dichloromethane phase was dried over magnesium sulfate and evaporated to give 0.35 g of a product used as such in the next step.

Title Compound

A mixture of the product of Preparation 3 (40.5 mg) in DMF (1 ml), Intermediate 49-2 (38.8 mg) in THF (1 ml) and pyridine (10 μl) was stirred at 55° C. during 20 hrs. The reaction mixture was then allowed to cool down to room temperature and HCl in isopropanol (5.5 M, 100 μl) and water (c.a. 1.5 ml) were added. The precipitate was filtered, washed with a 30:70 methanol/water solution and dried to yield 30 mg of the desired compound.

LCMS: Rt: 2.36 mn; M/z: 587; Purity: 90%

### Example 50

### N-[5-(3,5-Dichloro-2-hydroxy-phenyl)-[1,3,4]thiadiazol-2-yl]-N′-(3-methyl-4-[1,2,4]triazol-1-yl-phenyl)-terephthalamide

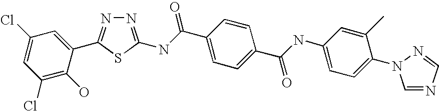

### 50A: 1-(2-Methyl-4-nitro-phenyl)-1H-[1,2,4]triazole

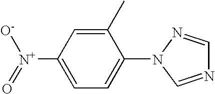

Intermediate 50-A was prepared by the operating procedure of intermediate 49A, starting from 2-fluoro-5-nitrotoluene.

¹H NMR (DMSO-d6): 9.15 (1H,s), 8.46 (1H,dd), 8.45(1H,s), 8.25(1H, dd), 7.75 (1H,d), 2.40 (3H,s)

### 50B: 3-Methyl-4-[1,2,4]triazol-1-yl-phenylamine

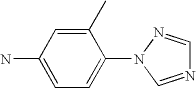

Intermediate 50B was prepared according to the operating procedure of Intermediate 49B starting from Intermediate 50-1

¹H NMR (DMSO-d6): 8.65 (1H,s), 8.15 (1H,s), 7.0 (1H,s), 6.55(1H, s), 6.50 (1H,dd), 5.45 (2H,broad signal), 1.95 (3H,s)

Title Compound:

A mixture of the product 7A (40.5 mg) in DMF (1 ml), intermediate 50B (37.6 mg) in THF (1 ml) and pyridine (10 □1) was stirred at 55° C. during 20 hrs. The reaction mixture was then allowed to cool down to room temperature and HCl in isopropanol (5.5 M, 100 □1) and water (c.a. 1.5 ml) were added. The precipitate was filtered, washed with a 30:70 methanol/water solution and dried to yield 25 mg of the desired compound.

LCMS: Rt: 2.25 mn; M/z: 567; Purity: 99%

### Example 51 to 73

Examples 51 to 73 were obtained using the following procedure:

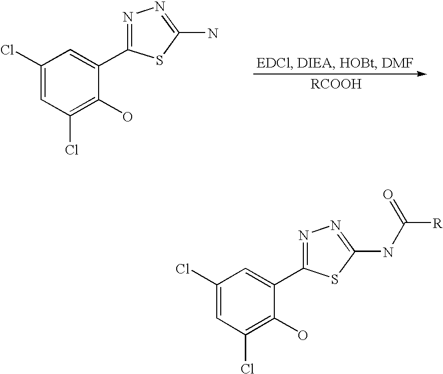

A mixture of 2-(5-Amino-[1,3,4]thiadiazol-2-yl)-4,6-dichloro-phenol (21 mg; 0.08 mmole), 1-hydroxybenzotriazole HOBT (13 mg, 0.096 mmole) and the corresponding acid (0.096 mmole) in DMF (1 ml) was stirred for 30 min at RT then a solution of 1-(3-dimethylaminopropyl)-3-ethylcarbodiimide EDCI (0.096 mmole) and diisopropylethylamine DIEA (0.096 mmole) in DMF (1 ml) was added. The reaction mixture was stirred at 100° C. for 20 hours then allowed to cool down to RT and HCl in isopropanol (5.5 M, 75 □1) was added. It was then loaded on a Strong Cation eXchange cartridge (SCX 150 mg conditioned with methanol). The cartridge was washed with THF (2.5 ml) and the filtrate was collected. Water (c.a. 2 ml) was added into it until a precipitate formed. The precipitate was filtered, washed with water then redissolved in DMF. The organic solvent was evaporated under reduced pressure to give the desired compound. When no precipitate had formed upon adding water, the reaction mixture was loaded on an extraction cartridge (Waters Oasis, HLB, 250 mg, prewashed with methanol, DMF and water). The cartridge was washed with a 20:20:60 saturated aqueous sodium bicarbonate/water/methanol solution then with water and eluted with DMF. Solvent was evaporated under reduced pressure from the elution fraction to yield the desired compound.

### Example 51

### 3-Bromo-N-[5-(3,5-dichloro-2-hydroxy-phenyl)-[1,3,4]thiadiazol-2-yl]-benzamide

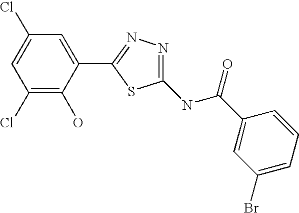

LCMS: Rt: 2.61 mn; M/z: 446; Purity: 98%

### Example 52

### 4-Chloro-N-[5-(3,5-dichloro-2-hydroxy-phenyl)-[1,3,4]thiadiazol-2-yl]-benzamide

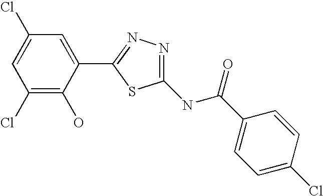

LCMS: Rt: 2.56 mn; M/z: 401; Purity: 92%

### Example 53

### 3,4-Dichloro-N-[5-(3,5-dichloro-2-hydroxy-phenyl)-[1.3.4]thiadiazol-2-yl]-benzamide

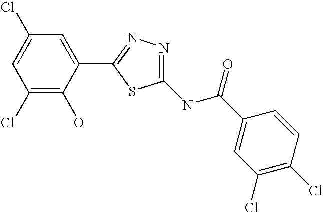

LCMS: Rt: 2.69 mn; M/z: 436; Purity: 92%

### Example 54

### N-[5-(3,5-Dichloro-2-hydroxy-phenyl)-[1,3,4]thiadiazol-2-yl]-3-iodo-benzamide

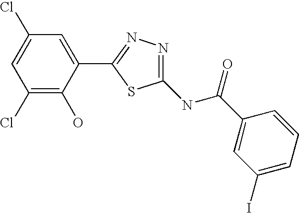

LCMS: Rt: 2.65 mn; M/z: 493; Purity: 95%

### Example 55

### N-[5-(3,5-Dichloro-2-hydroxy-phenyl)-[1,3,4]thiadiazol-2-yl]-4-methyl-3-nitro-benzamide

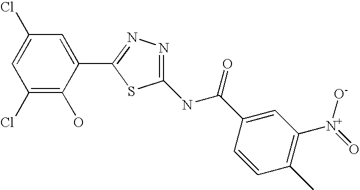

LCMS: Rt: 2.48 mn; M/z: 426; Purity: 95%

### Example 56

### Benzo[1,3]dioxole-5-carboxylic acid [5-(3,5-dichloro-2-hydroxy-phenyl)-[1,3,4]thiadiazol-2-yl]-amide

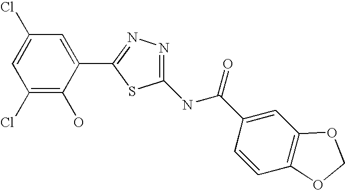

LCMS: Rt: 2.57 mn; M/z: 411; Purity: 86%

### Example 57

### 3-Benzoyl-N-[5-(3,5-dichloro-2-hydroxy-phenyl)-[1,3,4]thiadiazol-2-yl]-benzamide

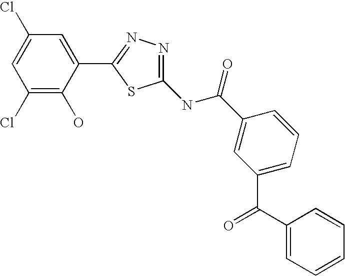

LCMS: Rt: 2.72 mn; M/z: 471; Purity: 83%

### Example 58

### N-[5-(3,5-Dichloro-2-hydroxy-phenyl)-[1,3,4]thiadiazol-2-yl]-4-pyrrol-1-yl-benzamide

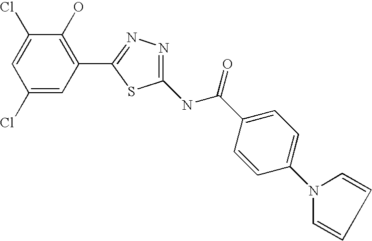

LCMS: Rt: 2.57 mn; M/z: 431; Purity: 86%

### Example 59

### 3-Bromo-N-[5-(3,5-dichloro-2-hydroxy-phenyl)-[1,3,4]thiadiazol-2-yl]-4-methyl-benzamide

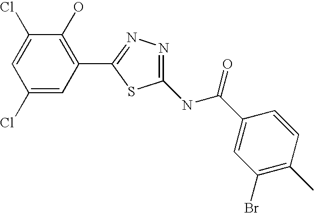

LCMS: Rt: 2.68 mn; M/z: 458; Purity: 85%

### Example 60

### 4-Bromo-N-[5-(3,5-dichloro-2-hydroxy-phenyl)-[1,3,4]thiadiazol-2-yl]-benzamide

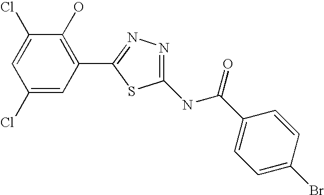

LCMS: Rt: 2.57 mn; M/z: 443; Purity: 85%

### Example 61

### N-[5-(3,5-Dichloro-2-hydroxy-phenyl)-[1,3,4]thiadiazol-2-yl]-3,4-diethoxy-benzamide

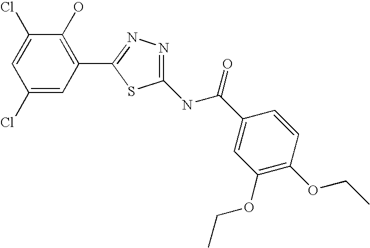

LCMS: Rt: 2.55 mn; M/z: 453; Purity: 95%

### Example 62

### 3-Bromo-N-[5-(3,5-dichloro-2-hydroxy-phenyl)-[1,3,4]thiadiazol-2-yl]-4-methoxy-benzamide

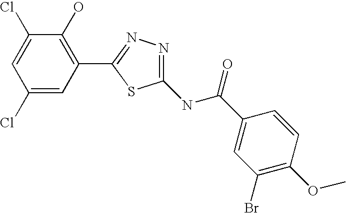

LCMS: Rt: 2.57 mn; M/z: 473; Purity: 88%

### Example 63

### 3-Chloro-N-[5-(3,5-dichloro-2-hydroxy-phenyl)-[1,3,4]thiadiazol-2-yl]-4-methoxy-benzamide

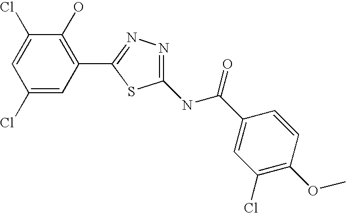

LCMS: Rt: 2.56 mn; M/z: 429; Purity: 91%

### Example 64

### N-[5-(3,5-Dichloro-2-hydroxy-phenyl)-[1,3,4]thiadiazol-2-yl]-3-fluoro-4-methyl-benzamide

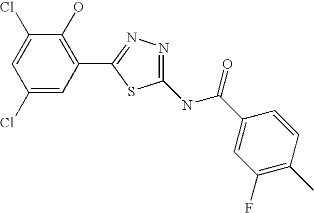

LCMS: Rt: 2.57 mn; M/z: 397; Purity: 100%

### Example 65

### N-[5-(3,5-Dichloro-2-hydroxy-phenyl)-[1,3,4]thiadiazol-2-yl]-4-iodo-benzamide

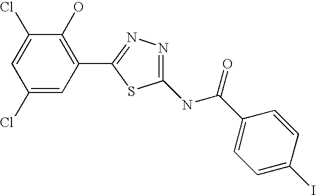

LCMS: Rt: 2.59 mn; M/z: 491; Purity: 93%

### Example 66

### 4-Chloro-N-[5-(3,5-dichloro-2-hydroxy-phenyl)-[1,3,4]thiadiazol-2-yl]-3-methyl-benzamide

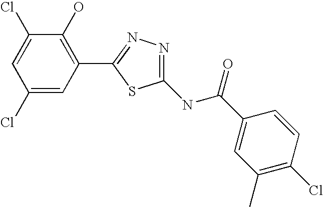

LCMS: Rt: 2.64 mn; M/z: 413; Purity: 93%

### Example 67

### 4-Bromo-N-[5-(3,5-dichloro-2-hydroxy-phenyl)-1,3,4]thiadiazol-2-yl]-3-methyl-benzamide

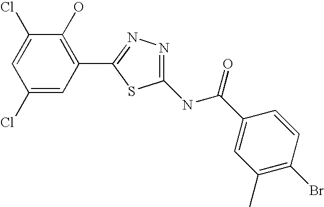

LCMS: Rt: 2.67 mn; M/z: 457; Purity: 84%

### Example 68

### N-[5-(3,5-Dichloro-2-hydroxy-phenyl)-[1,3,4]thiadiazol-2-yl]-4-propyl-benzamide

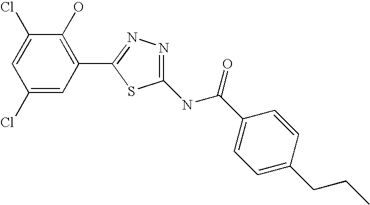

LCMS: Rt: 2.68 mn; M/z: 407; Purity: 87%

### Example 69

### N-[5-(3,5-Dichloro-2-hydroxy-phenyl)-[1,3,4]thiadiazol-2-yl]-4-ethoxy-benzamide

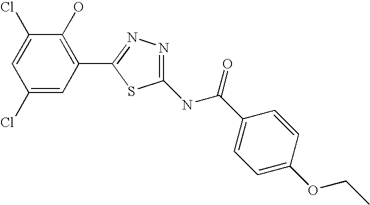

LCMS: Rt: 2.55 mn; M/z: 409; Purity: 93%

### Example 70

### N-[5-(3,5-Dichloro-2-hydroxy-phenyl)-[1,3,4]thiadiazol-2-yl]-4-methoxy-3-nitro-benzamide

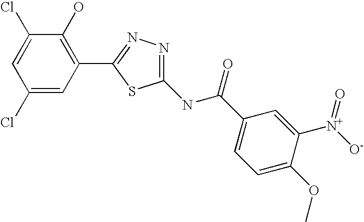

LCMS: Rt: 2.39 mn; M/z: 440; Purity: 91%

### Example 71

### {4-[5-(3,5-Dichloro-2-hydroxy-phenyl)-[1,3,4]thiadiazol-2-ylcarbamoyl]-phenyl}-carbamic acid tert-butyl ester

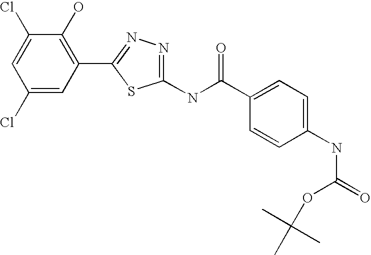

LCMS: Rt: 2.57 mn; M/z: 480; Purity: 91%

### Example 72

### 4-Butoxy-N-[5-(3,5-dichloro-2-hydroxy-phenyl)-[1,3,4thiadiazol-2-yl]-benzamide

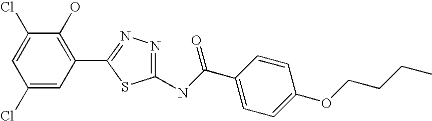

LCMS: Rt: 2.84 mn; M/z: 438; Purity: 88%

### Example 73

### N-[5-(3,5-Dichloro-2-hydroxy-phenyl)-[1,3,4]thiadiazol-2-yl]-3,5-dihydroxy-benzamide

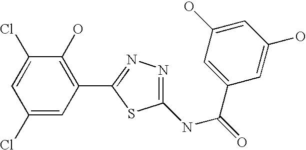

LCMS: Rt: 2.12 mn; M/z: 398; Purity: 88%

### Example 74

### Pyridine-2,5-dicarboxylic acid 5-{[5-(3,5-dichloro-2-hydroxy-phenyl)-[1,3,4]thiadiazol-2-yl]-amide}2-phenylamide

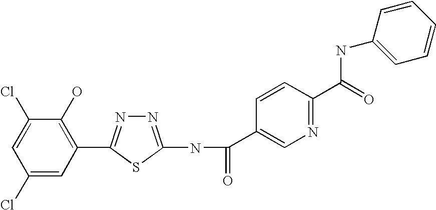

### 74A: 6-Phenylcarbamoyl-nicotinic acid methyl ester

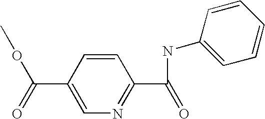

0.33 g of pyridine-2,5-dicarboxylic acid 5-methyl ester were dissolved in DMF and 0.17 g aniline and 0.38 g HOBt were added. 0.4 ml DIEA were then added and the reaction mixture was stirred at 60° C. for 2 hours. After return to RT, dilute HCl was added and the mixture was filtrated to give 0.41 g of the expected product.

¹H NMR (DMSO-d6): 10.95 (1H,s), 9.20 (1H,s), 8.55 (1H,d), 8.30(1H,d), 7.95(2H, m), 7.35 (2H,m), 7.20 (1H,dd), 3.95 (3H,s)

### 74B: 6-Phenylcarbamoyl-nicotinic acid

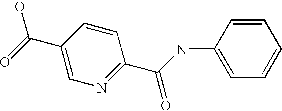

410 mg of Intermediate 74A was dissolved in a THF/methanol mixture and 5 ml of 1N NaOH was added. The mixture was stirred at RT for 1 day and the solvents were evaporated. Concentrated HCl was added and the precipitate was filtered and washed with acetone to give 280 mg of the expected product.

¹H NMR (DMSO-d6): 10.85 (1H,s), 9.18 (1H,s), 8.55 (1H,dd), 8.30(1H,d), 8.0 (2H, d), 7.40 (2H,dd), 7.15 (1H,dd).

Title Compound

0.3 g of the product of 1B was dissolved in DMF and 0.15 g of Intermediate 74B, 0.27 g of HOBT and 0.4 ml of DIEA were added. The mixture was stirred at RT for 30 mn and 0.35 g of EDCI were then added. The reaction mixture was heated at 65° C. for 3 hours, water was added and the obtained precipitate was washed by dilute HCl and acetone to give 0.120 g of a product melting over 330° C.

Microanalysis: theory(%): C, 51.9; H, 2.69; N, 14.4; obtained (%): C, 51.7; H, 2.79; N, 14.4.

### Example 75

### Pyridine-2,5-dicarboxylic acid 2-{[5-(3,5-dichloro-2-hydroxy-phenyl)-[1,3,4]thiadiazol-2-yl]-amide}5-phenylamide

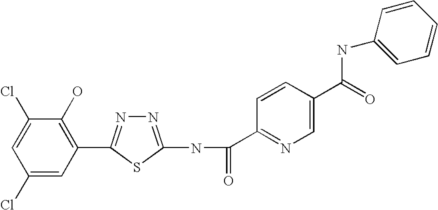

### 75A: 6-[5-(3,5-Dichloro-2-hydroxy-phenyl)-[1,3,4]thiadiazol-2-ylcarbamoyl]-nicotinic acid methyl ester

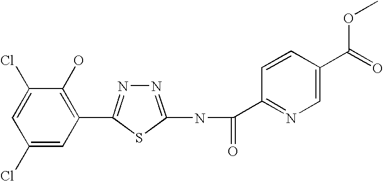

1 g of the product of Preparation 1B was dissolved in DMF and 0/7 g of pyridine-2,5-dicarboxylic acid 5-methyl ester, 0.8 g of HOBT and 0.85 ml of DIEA were added. The mixture was stirred at RT for 30 mn and 0.9 g of EDCI were then added. The reaction mixture was heated at 65° C. for 3 hours, water was added and the obtained precipitate was washed by dilute HCl and acetone to give 1.2 g of a product melting at 301° C.

Microanalysis: theory(%): C, 45.2; H, 2.37; N, 13.2; obtained (%): C, 44.9; H, 2.26; N, 13.0.

### 75B: 6-[5-(3,5-Dichloro-2-hydroxy-phenyl)-[1,3,4]thiadiazol-2-ylcarbamoyl]-nicotinic acid

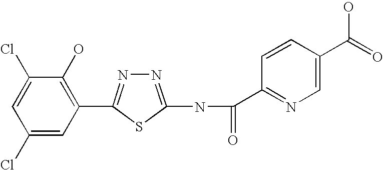

800 mg of Intermediate 75A was dissolved in a mixture of methanol and THF and 0.800 ml of concentrated NaOH were added. The mixture was heated at 50° C. for 24 hours, the solvents were evaporated and concentrated HCl was added. The resulting precipitate was filtered and washed with acetone to give 720 mg of a product melting at 260° C.

Title compound:

600 mg of Intermediate 75B was dissolved in DMF and 270 mg HOBt, 0.400 ml DIEA and 150 mg of aniline were added. The reaction mixture was stirred at RT for 20 mn and 350 mg EDCI were added. The reaction mixture was stirred for 3 hours at 60° C. After return to RT, water was added and the obtained precipitate was filtered out and washed with acetone to give 310 mg of a product melting at 267° C.

Microanalysis: theory(%): C, 51.9; H, 2.69; N, 14.4; obtained (%): C, 51.7; H, 2.83; N, 14.5.

### Example 76

### 4-Benzoylamino-N-[5-(3,5-dichloro-2-hydroxy-phenyl)-[1,3,4]thiadiazol-2-yl]-benzamide

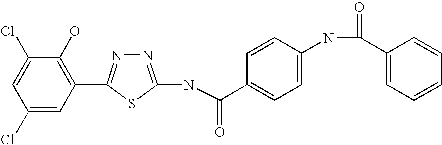

0.300 g of the product of 1B was dissolved in DMF and 0.28 g of 4-aminobenzoyl-benzoic acid (prepared according to J. Med. Chem., 1988, p. 590-603) 0.24 g of HOBT and 0.25 ml of DIEA were added. The mixture was stirred at RT for 30 mn and 0.27 g of EDCI were then added. The reaction mixture was heated at 65° C. for 3 hours, water was added and the obtained precipitate was washed by methanol to give 0.300 g of a product melting at 301° C.

Microanalysis: theory(%): C, 54.4; H, 2.91; N, 11.5; obtained (%): C, 54.4; H, 2.86; N, 11.2.

1. A method of treating a disorder associated with high levels of PAI-1 comprising administering to a patient in need thereof a therapeutically effective amount of at least one compound of formula (I), 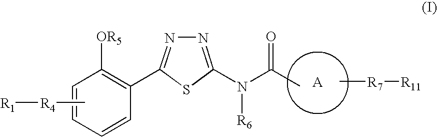
or a pharmaceutically-acceptable salt, prodrug, stereoisomer or solvate thereof, wherein:
A is aryl or heteroaryl;
R₁-R₄ and R₇—R₁₁ are independently selected from hydrogen, halogen, nitro, cyano, alkyl, substituted alkyl, aryl, heteroaryl, cycloalkyl, heterocyclo, —OR₁₂, —SR₁₂, —OC(═O)R₁₂, —C(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₅CO₂R₁₂, —NR₁₅C(═O)NR₁₃R₁₄;
or any two of R₁—R₄ and R₇—R₁₁ located on neighboring atoms of the ring to which they are attached may be taken together to form a fused ring system in combination with the ring, wherein the fused ring system may be optionally further substituted;
R₅ is hydrogen, C₁₋₆alkyl or substituted C₁₋₆alkyl;
R₆ is hydrogen or C₁₋₆alkyl; and
R₁₂, R₁₃, R₁₄ and R₁₅ are independently selected from hydrogen, alkyl, subtituted alkyl, aryl, heteroaryl, cycloalkyl and heterocyclo, wherein each instance of R₁₂, R₁₃, R₁₄ and/or R₁₅ is selected independently.

2. The method according to claim 1, comprising administering to a patient in need thereof a therapeutically effective amount of at least one compound having the formula (Ia), 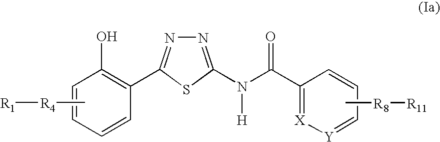
or a pharmaceutically-acceptable salt, prodrug, stereoisomer or solvate thereof, wherein:
X and Y are independently carbon or nitrogen;
R₁ to R₄ are independently selected from hydrogen, halogen, nitro, cyano, alkyl, substituted alkyl, —OR₁₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₂, —NR₁₃C(═O)R₁₂, —NR₁₅CO₂R₁₂, NR₁₅C(═O)NR₁₃R₁₄;
R₈-R₁₁ are independently selected from hydrogen, halogen, nitro, cyano, alkyl, substituted alkyl, aryl, heteroaryl, cycloalkyl, heterocyclo, —OR,₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₅C(═O)R₁₂, —NR₁₅CO₂R₁₂, —NR₁₅C(═O)NR₁₃R₁₄; and
or any two of R₈—R₁₁ located on neighboring atoms of the ring may be taken together to form a fused ring system in combination with the ring, said fused ring system being optionally substituted.

3. A method according to claim 2 comprising administering to a patient in need thereof a therapeutically effective amount of at least one compound having the formula (Ib), 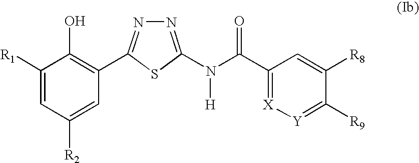
or a pharmaceutically-acceptable salt, prodrug, stereoisomer or solvate thereof, wherein:
R¹ and R₂ are independently selected from halogen, nitro, cyano, C₁₋₆alkyl and C₁₋₆substituted alkyl; and
R₈ and R₉ are independently selected from hydrogen, halogen, nitro, cyano, alkyl, substituted alkyl, aryl, heteroaryl, heterocyclo, —OR₁₂, —SR₁₂, —C(═O)R₁₂, 13 CO₂R₁₂, —C(═O)NR₁₃R₁₄, —NR₁₃R₁₄, —S(═O)R₁₂, —SO₂R₁₂, —SO₂NR₁₃R₁₄, —NR₁₅CO₂R₁₂, and —NR₁₅C(═O)R₁₂;
or R₈ and R₉ may be taken together to form a fused ring system in combination with the ring, said fused ring system being optionally substituted.

4. A method according to claim 2 comprising administering to a patient in need thereof a therapeutically effective amount of at least one compound having the formula (Ia), or a pharmaceutically acceptable salt, prodrug, stereoisomer, or solvate thereof, in which:
R₁₂ and R₁₃ are independently selected from hydrogen, C₁₋₆alkyl, substituted C₁₋₆alkyl, aryl and heteroaryl; and
R₁₄ and R₁₅ are independently selected from hydrogen, C₁₋₆alkyl and, substituted C₁₋₆alkyl.

5. A method according to claim 4 comprising administering to a patient in need thereof a therapeutically effective amount of at least one compound having the formula (Ic), 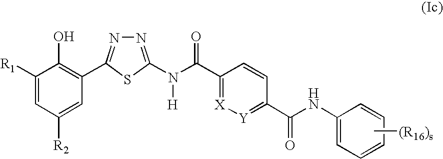
or a pharmaceutically-acceptable salt, prodrug, stereoisomer or solvate thereof, wherein:
R₁₆ is selected from hydrogen, halogen, nitro, cyano, C₁₋₆alkyl, substituted C₁₋₆alkyl, —OR₁₇, —SR₁₇, C(O)R₁₇, —S(═O)R₁₇, —CO₂R₁₇, —C(═O)NR₁₈R₁₉, —NR₁₈R₁₉, —S(═O)R₁₇, —SO₂R₁₇, —SO₂NR₁₈R₁₉ and —NR₂₀C(═O)R₁₇ aryl and heteroaryl;
R₁₇ is hydrogen halogen, C₁₋₆alkyl, aryl or heteroaryl;
R₁₈, and R₁₉ are independently selected from hydrogen, C₁₋₆alkyl, aryl or heteroaryl;
s is an integer selected from 1-3; and
wherein each instance of R₁₆—R₁₉ is selected independently.

6. A method according to claim 1, comprising administering to a patient in need thereof a therapeutically effective amount of at least one compound of formula (I), or a pharmaceutically-acceptable salt, prodrug, stereoisomer or solvate thereof, in which
R₁ and R₂ are independently halogen.

7. A method according to claim 1 comprising administering to a patient in need thereof a therapeutically effective amount of at least one compound having the formula (Ia), or a pharmaceutically acceptable salt, prodrug, stereoisomer, or solvate thereof, in which X and Y are independently carbon or nitrogen; provided that X and Y are not both nitrogen.

8. A compound having the formula (Ia), 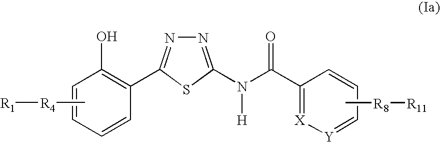
or a pharmaceutically-acceptable salt, prodrug, stereoisomer or solvate thereof, wherein:
X and Y are independently carbon or nitrogen;
R₁ to R₄ are independently selected from hydrogen, halogen, nitro, cyano, alkyl, substituted alkyl, —OR₁₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₃C(═O)R₁₂, —NR₁₅CO₂R₁₂, NR₁₅C(═O)NR₁₃R₁₄;
R₈-R₁₁ are independently selected from hydrogen, halogen, nitro, cyano, alkyl, substituted alkyl, aryl, heteroaryl, cycloalkyl, heterocyclo, —OR₁₂, —SR₁₂, —C(═O)R₁₂, —OC(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₅C(═O)R₁₂, —NR₁₅CO₂R₁₂, —NR₁₅C(═O)NR₁₃R₁₄; and
or any two of R₈—R₁₁ located on neighboring atoms of the ring may be taken together to form a fused ring system in combination with the ring, said fused ring system being optionally substituted; and
R₁₂, R₁₃, R₁₄ and R₁₅ are independently selected from hydrogen, alkyl, substituted alkyl, aryl, heteroaryl, cycloalkyl and heterocyclo, wherein each instance of R₁₂, R₁₃, R₁₄ and/or R₁₅ is selected independently.

9. A compound according to claim 8 having the formula (Ib), 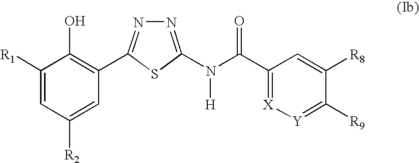
or a pharmaceutically-acceptable salt, prodrug, stereoisomer or solvate thereof, wherein:
R₁ and R₂ are independently selected from halogen, nitro, cyano, C₁₋₆alkyl and C₁₋₆substituted alkyl; and
R₈ and R₉ are independently selected from hydrogen, halogen, nitro, cyano, alkyl, substituted alkyl, aryl, heteroaryl, heterocyclo, —OR₁₂, —SR₁₂, —C(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅CO₂R₁₂, and —NR₁₅C(═O)R₁₂;
or R₈ and R₉ may be taken together to form a fused ring system in combination with the ring, said fused ring system being optionally substituted.

10. A compound according to claim 9 or a pharmaceutically acceptable salt, prodrug or solvate thereof, in which:
R₁₂ and R₁₃ are independently selected from hydrogen, C₁₋₆alkyl, substituted C₁₋₆alkyl, aryl and heteroaryl; and
R₁₄ and R₁₅ are independently selected from hydrogen, C₁₋₆alkyl and substituted C₁₋₆alkyl.

11. A compound according to claim 10, having the formula (Ic), 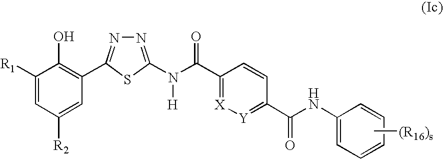
or a pharmaceutically-acceptable salt, prodrug, stereoisomer or solvate thereof, wherein:
R₁₆ is selected from hydrogen, halogen, nitro, cyano, C₁₋₆alkyl, substituted C₁₋₆alkyl, —OR₁₇, —SR₁₇, —C(O)R₁₇, —S(═O)R₁₇, —CO₂R₁₇, —C(═O)NR₁₈R₁₉, —NR₁₈R₁₉, —S(═O)R₁₇, —SO₂R₁₇, —SO₂NR₁₈R₁₉, —NR₂₀C(═O)R₁₇, aryl and heteroaryl;
R₁₇ is hydrogen halogen, C₁₋₆alkyl, aryl or heteroaryl;
R₁₈, R₁₉ and R₂₀ are independently selected from hydrogen, C₁₋₆alkyl, substituted C₁₋₆alkyl, aryl or heteroaryl;
s is an integer selected from 1-3; and
wherein each instance of R₁₆—R₁₉ is selected independently.

12. A compound of claim 8, or a pharmaceutically-acceptable salt, prodrug, stereoisomer or solvate thereof, in which R₁ and R₂ are independently halogen.

13. A compound according to claim 8 or a pharmaceutically acceptable salt, prodrug or solvate thereof, in which:
X and Y are independently carbon or nitrogen; provided that X and Y are not both nitrogen.

14. A compound selected from
(i) N-[5-(3,5-Dichloro-2-hydroxy-phenyl)-[1,3,4]thiadiazol-2-yl]-terephthalamic acid methyl ester;
N-[5-(3,5-Dichloro-2-hydroxy-phenyl)-[1,3,4]thiadiazol-2-yl]-terephthalamic acid;
N-[5-(3,5-Dichloro-2-hydroxy-phenyl)-[1,3,4]thiadiazol-2-yl]-N′-phenyl-terephthalamide;
N-[5-(3,5-Dichloro-2-hydroxy-phenyl)-[1,3,4]thiadiazol-2-yl]-N′-(4-phenoxy-phenyl)-terephthalamide;
4-(4-{4-[5-(3,5-Dichloro-2-hydroxy-phenyl)-[1,3,4]thiadiazol-2-ylcarbamoyl]-benzoylamino}-phenoxy)-benzoic acid methyl ester;
4-(4-{4-[5-(3,5-Dichloro-2-hydroxy-phenyl)-[1,3,4]thiadiazol-2-ylcarbamoyl]-benzoylamino}-phenoxy)-benzoic acid;
3-{4-[5-(3,5-Dichloro-2-hydroxy-phenyl)-[1,3,4]thiadiazol-2-ylcarbamoyl]-benzoylamino}-benzoic acid ethyl ester;
N-(4-Chloro-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-fluoro-phenyl)-terephthalamide;
N-[5-(3,5-Dichloro-2-hydroxy-phenyl)-[1,3,4]thiadiazol-2-yl]-N′-(4-fluoro-phenyl)-terephthalamide;
N-[5-(3,5-Dichloro-2-hydroxy-phenyl)-[1,3,4]thiadiazol-2-yl]-N′-(9-ethyl-9H-carbazol-3-yl)-terephthalamide;
N-[5-(3,5-Dichloro-2-hydroxy-phenyl)-[1,3,4]thiadiazol-2-yl]-N′-(4-pentyloxy-phenyl)-terephthalamide;
N-[5-(3,5-Dichloro-2-hydroxy-phenyl)-[1,3,4]thiadiazol-2-yl]-N′-(4-phenylamino-phenyl)-terephthalamide;
2-{4-[5-(3,5-Dichloro-2-hydroxy-phenyl)-[1,3,4]thiadiazol-2-ylcarbamoyl]-benzoylamino}-benzoic acid methyl ester;
N-[5-(3,5-Dichloro-2-hydroxy-phenyl)-[1,3,4]thiadiazol-2-yl]-N′-p-tolyl-terephthalamide;
N-(2-Benzoyl-4-chloro-phenyl)-N′-[5-(3,5-dichloro-2-hydroxy-phenyl)-[1,3,4]thiadiazol-2-yl]-terephthalamide;
2-{4-[5-(3,5-Dichloro-2-hydroxy-phenyl)-[1,3,4]thiadiazol-2-ylcarbamoyl]-benzoylamino}-4,5-dimethoxy-benzoic acid methyl ester;
2-{4-[5-(3,5-Dichloro-2-hydroxy-phenyl)-[1,3,4]thiadiazol-2-ylcarbamoyl]-benzoylamino}-terephthalic acid dimethyl ester;
N-[2-(4-Chloro-benzoyl)-phenyl]-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(4-ethoxy-phenyl)-terephthalamide;
N-(3-Chloro-4-methoxy-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(2-methoxy-5-nitro-phenyl)-terephthalamide;
N-Biphenyl-4-yl-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(2-nitro-phenyl)-terephthalamide;
N-(3-Chloro-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(2,5-difluoro-phenyl)-terephthalamide;
N-[5-(3,5-Dichloro-2-hydroxy-phenyl)-[1,3,4]thiadiazol-2-yl]-N′-(3,5-difluoro-phenyl)-terephthalamide;
N-[5-(3,5-Dichloro-2-hydroxy-phenyl)-[1,3,4]thiadiazol-2-yl]-N′-(3,4-dichloro-phenyl)-terephthalamide;
N-(3-Cyano-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(4-hydroxy-phenyl)-terephthalamide;
2-{4-[5-(3,5-Dichloro-2-hydroxy-phenyl)-[1,3,4]thiadiazol-2-ylcarbamoyl]-benzoylamino}-4,5-dimethoxy-benzoic acid;
N-(4-Benzyloxy-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-methylsulfanyl-phenyl)-terephthalamide;
N-[5-(3,5-Dichloro-2-hydroxy-phenyl)-[1,3,4]thiadiazol-2-yl]-N′-(4-methylsulfanyl-phenyl)-terephthalamide;
N-[5-(3,5-Dichloro-2-hydroxy-phenyl)-[1,3,4]thiadiazol-2-yl]-N′-(9H-fluoren-1-yl)-terephthalamide;
N-(4-Carbamoyl-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-nitro-phenyl)-terephthalamide;
N-[4-(4-Bromo-1-methyl-1H-pyrazol-3-yl)-phenyl]-N′-[5-(3,5-dichloro-2-hydroxy-phenyl)-[1,3,4]thiadiazol-2-yl]-terephthalamide;
5-(4-{4-[5-(3,5-Dichloro-2-hydroxy-phenyl)-[1,3,4]thiadiazol-2-ylcarbamoyl]-benzoylamino}-phenyl)-2-methyl-furan-3-carboxylic acid ethyl ester;
N-[5-(3,5-Dichloro-2-hydroxy-phenyl)-[1,3,4]thiadiazol-2-yl]-N′-[4-(2-methyl-2H-pyrazol-3-yl)-phenyl]-terephthalamide;
N-[4-(5-tert-Butyl-[1,2,3]triazol-1-yl)-phenyl]-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-[4-(2H-tetrazol-5-yl)-phenyl]-terephthalamide;
N-[5-(3,5-Dichloro-2-hydroxy-phenyl)-[1,3,4]thiadiazol-2-yl]-N′-(4-imidazol-1-yl-phenyl)-terephthalamide;
N-[5-(3,5-Dichloro-2-hydroxy-phenyl)-[1,3,4]thiadiazol-2-yl]-N′-(4-[1,2,4]triazol-1-yl-phenyl)-terephthalamide;
Hydrochloride of N-[5-(3,5-Dichloro-2-hydroxy-phenyl)-[1,3,4]thiadiazol-2-yl]-N′-(4-[1,2,3]triazol-2-yl-phenyl)-terephthalamide;
Hydrochloride of N-[5-(3,5-Dichloro-2-hydroxy-phenyl)-[1,3,4]thiadiazol-2-yl]-N′-(4-[1,2,3]triazol-1-yl-phenyl)-terephthalamide;
N-[5-(3,5-Dichloro-2-hydroxy-phenyl)-[1,3,4]thiadiazol-2-yl]-N′-[4-(2-methyl-2H-tetrazol-5-yl)-phenyl]-terephthalamide;
N-(3-Chloro-4-[1,2,4]triazol-1-yl-phenyl)-N′-[5-(3,5-dichloro-2-hydroxy-phenyl)-[1,3,4]thiadiazol-2-yl]-terephthalamide;
N-[5-(3,5-Dichloro-2-hydroxy-phenyl)-[1,3,4]thiadiazol-2-yl]-N′-(3-methyl-4-[1,2,4]triazol-1-yl-phenyl)-terephthalamide;
3-Bromo-N-[5-(3,5-dichloro-2-hydroxy-phenyl)-[1,3,4]thiadiazol-2-yl]-benzamide;
4-Chloro-N-[5-(3,5-dichloro-2-hydroxy-phenyl)-[1,3,4]thiadiazol-2-yl]-benzamide;
3,4-Dichloro-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-3-iodo-benzamide;
N-[5-(3,5-Dichloro-2-hydroxy-phenyl)-[1,3,4]thiadiazol-2-yl]-4-methyl-3-nitro-benzamide′
Benzo[1,3]dioxole-5-carboxylic acid [5-(3,5-dichloro-2-hydroxy-phenyl)-[1,3,4]thiadiazol-2-yl]-amide;
3-Benzoyl-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-4-pyrrol-1-yl-benzamide;
3-Bromo-N-[5-(3,5-dichloro-2-hydroxy-phenyl)-[1,3,4]thiadiazol-2-yl]-4-methyl-benzamide;
4-Bromo-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-3,4-diethoxy-benzamide;
3-Bromo-N-[5-(3,5-dichloro-2-hydroxy-phenyl)-[1,3,4]thiadiazol-2-yl]-4-methoxy-benzamide;
3-Chloro-N-[5-(3,5-dichloro-2-hydroxy-phenyl)-[1,3,4]thiadiazol-2-yl]-4-methoxy-benzamide;
N-[5-(3,5-Dichloro-2-hydroxy-phenyl)-[1,3,4]thiadiazol-2-yl]-3-fluoro-4-methyl-benzamide;
N-[5-(3,5-Dichloro-2-hydroxy-phenyl)-[1,3,4]thiadiazol-2-yl]-4-iodo-benzamide;
4-Chloro-N-[5-(3,5-dichloro-2-hydroxy-phenyl)-[1,3,4]thiadiazol-2-yl]-3-methyl-benzamide
4-Bromo-N-[5-(3,5-dichloro-2-hydroxy-phenyl)-[1,3,4]thiadiazol-2-yl]-3-methyl-benzamide;
N-[5-(3,5-Dichloro-2-hydroxy-phenyl)-[1,3,4]thiadiazol-2-yl]-4-propyl-benzamide;
N-[5-(3,5-Dichloro-2-hydroxy-phenyl)-[1,3,4]thiadiazol-2-yl]-4-ethoxy-benzamide;
N-[5-(3,5-Dichloro-2-hydroxy-phenyl)-[1,3,4]thiadiazol-2-yl]-4-methoxy-3-nitro-benzamide;
{4-[5-(3,5-Dichloro-2-hydroxy-phenyl)-[1,3,4]thiadiazol-2-ylcarbamoyl]-phenyl}-carbamic acid tert-butyl ester;
4-Butoxy-N-[5-(3,5-dichloro-2-hydroxy-phenyl)-[1,3,4]thiadiazol-2-yl]-benzamide;
N-[5-(3,5-Dichloro-2-hydroxy-phenyl)-[1,3,4]thiadiazol-2-yl]-3,5-dihydroxy-benzamide;
Pyridine-2,5-dicarboxylic acid 5-{[5-(3,5-dichloro-2-hydroxy-phenyl)-[1,3,4]thiadiazol-2-yl]-amide}2-phenylamide;
Pyridine-2,5-dicarboxylic acid 2-{[5-(3,5-dichloro-2-hydroxy-phenyl)-[1,3,4]thiadiazol-2-yl]-amide}5-phenylamide;
4-Benzoylamino-N-[5-(3,5-dichloro-2-hydroxy-phenyl)-[1,3,4]thiadiazol-2-yl]-benzamide; or
(ii) a pharmaceutically acceptable salt, prodrug, stereoisomer or solvate of (i) thereof.

15. A pharmaceutical composition, comprising a pharmaceutically acceptable carrier and a therapeutically effective amount of a compound of claim 8.

16. A method according to claim 1, wherein the disorder associated with high levels of PAI-1 is a thromboembolic disorder.

17. A method according to claim 16 wherein the thromboembolic disorder is selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

18. A method for treating a thromboembolic disorder, comprising: administering to a patient in need thereof a therapeutically effective amount of a first and second therapeutic agent, wherein the first therapeutic agent is a compound having formula (I) or a pharmaceutically acceptable salt or hydrate thereof and the second therapeutic agent is at least one agent selected from a second PAI-1 inhibitor, a factor Xa inhibitor, an anti-coagulant agent, an anti-platelet agent, a thrombin inhibiting agent, a thrombolytic agent, and a fibrinolytic agent wherein formula (I) is 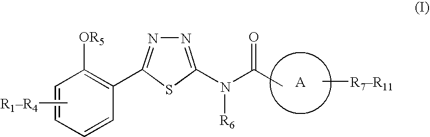
or a pharmaceutically-acceptable salt, prodrug, stereoisomer or solvate thereof, wherein:
A is aryl or heteroaryl;
R₁--R₄ and R₇-R₁₁ are independently selected from hydrogen, halogen, nitro, cyano, alkyl, substituted alkyl, aryl, heteroaryl, cycloalkyl, heterocyclo, —OR₁₂, —SR₁₂, —C(═O)R₁₂, —C(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₃C(═O)R₁₂, —NR₁₅CO₂R₁₂, —NR₁₅C(═O)NR₁₃R₁₄;
or any two of R₁-R₄ and R₇-R₁₁ located on neighboring atoms of the ring to which they are attached may be taken together to form a fused ring system in combination with the ring, wherein the fused ring system may be optionally further substituted;
R₅ is hydrogen, C₁₋₆alkyl or substituted C₁₋₆alkyl;
R₆ is hydrogen or C₁₋₆alkyl; and
R₁₂, R₁₃, R₁₄ and R₁₅ are independently selected from hydrogen, alkyl, subtituted alkyl, aryl, heteroaryl, cycloalkyl and heterocyclo, wherein each instance of R₁₂, R₁₃, R₁₄ and/or R₁₅ is selected independently.

19. A method according to claim 18 wherein the second therapeutic agent is at least one agent selected from warfarin, unfractionated heparin, low molecular weight heparin, synthetic pentasaccharide, hirudin, argatrobanas, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, disulfatohirudin, tissue plasminogen activator, modified tissue plasminogen activator, anistreplase, urokinase, and streptokinase.

20. The method according to claim 19, wherein the second therapeutic agent is at least one anti-platelet agent.

21. The method according to claim 20, wherein the anti-platelet agent is aspirin and clopidogrel.

22. The method according to claim 21, wherein the anti-platelet agent is clopidogrel.

23. An article of manufacture, comprising:
(a) a first container;
(b) a pharmaceutical composition located within the first container, wherein the composition, comprises: a first therapeutic agent, comprising: a compound of any one of formula (I), or a pharmaceutically acceptable salt or hydrate form thereof; and
(c) a package insert stating that the pharmaceutical composition can be used for the treatment of a thromboembolic disorder
wherein formula (I) is 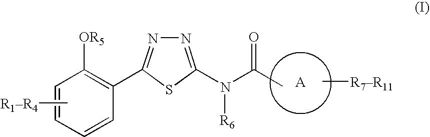
or a pharmaceutically-acceptable salt, prodrug, stereoisomer or solvate thereof, wherein:
A is aryl or heteroaryl;
R₁-R₄ and R₇-R₁₁ are independently selected from hydrogen, halogen, nitro, cyano, alkyl, substituted alkyl, aryl, heteroaryl, cycloalkyl, heterocyclo, —OR₁₂, —SR₁₂, —OC(═O)R₁₂, —C(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₃C(═O)R₁₂, —NR₁₅CO₂R₁₂, —NR₁₅C(═O)NR₁₃R₁₄;
or any two of R₁-R₄ and R₇-R₁₁ located on neighboring atoms of the ring to which they are attached may be taken together to form a fused ring system in combination with the ring, wherein the fused ring system may be optionally further substituted;
R₅ is hydrogen, C₁₋₆alkyl or substituted C₁₋₆alkyl;
R₆ is hydrogen or C₁₋₆alkyl; and
R₁₂, R₁₃, R₁₄ and R₁₅ are independently selected from hydrogen, alkyl, subtituted alkyl, aryl, heteroaryl, cycloalkyl and heterocyclo, wherein each instance of R₁₂, R₁₃, R₁₄ and/or R₁₅ is selected independently.

24. A article of manufacture according to claim 23, further comprising:
(d) a second container;
wherein components (a) and (b) are located within the second container and component (c) is located within or outside of the second container.

25. A compound of claim 8, or a pharmaceutically acceptable salt or hydrate form thereof, for use in therapy.

26. Use of a compound having formula (I), for the manufacture of a medicament for the treatment of a thromboembolic disorder wherein formula (I) is 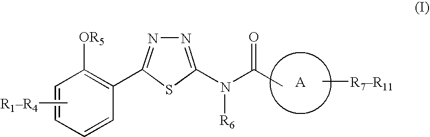
or a pharmaceutically-acceptable salt, prodrug, stereoisomer or solvate thereof, wherein:
A is aryl or heteroaryl;
R₁-R₄ and R₇-R₁₁ are independently selected from hydrogen, halogen, nitro, cyano, alkyl, substituted alkyl, aryl, heteroaryl, cycloalkyl, heterocyclo, —OR₁₂, —SR₁₂, —OC(═O)R₁₂, —C(═O)R₁₂, —CO₂R₁₂, —C(═O)NR₁₃R₁₄, —NR₁₃R₁₄, —S(═O)R₁₂, —SO₂R₁₂, —SO₂NR₁₃R₁₄, —NR₁₅SO₂NR₁₃R₁₄, —NR₁₅SO₂R₁₂, —NR₁₃C(═O)R₁₂, —NR₁₅CO₂R₁₂, —NR₁₅C(═O)NR₁₃R₁₄;
or any two of R₁—R₄ and R₇—R₁₁ located on neighboring atoms of the ring to which they are attached may be taken together to form a fused ring system in combination with the ring, wherein the fused ring system may be optionally further substituted;
R₅ is hydrogen, C₁₋₆alkyl or substituted C₁₋₆alkyl;
R₆ is hydrogen or C₁₋₆alkyl; and
R₁₂, R₁₃, R₁₄ and R₁₅ are independently selected from hydrogen, alkyl, subtituted alkyl, aryl, heteroaryl, cycloalkyl and heterocyclo, wherein each instance of R₁₂, R₁₃, R₁₄ and/or R₁₅ is selected independently.
